(19) United States
(12) Patent Application Publication
(10) Pub. No.: US 2022/0090063 A1
(43) Pub. Date: Mar. 24, 2022

(54) CRISPR/CAS9 IN VITRO KIT

(71) Applicant: WISYS TECHNOLOGY FOUNDATION, INC., Madison, WI (US)

(72) Inventor: Tiffany Rose Hoage, Menomonie, WI (US)

(21) Appl. No.: 17/482,884

(22) Filed: Sep. 23, 2021

Related U.S. Application Data
(60) Provisional application No. 63/082,192, filed on Sep. 23, 2020.

(51) Int. Cl. C12N 15/11; C12N 9/22; B01L 7/00; C12N 15/90; C12Q 1/6806; B01L 3/00; B04B 5/04; G01N 27/447

(52) U.S. Cl. CPC C12N 15/11; C12N 9/22; B01L 7/00; C12N 15/902; C12Q 1/6806; B01L 3/52; B01L 2300/123; B04B 5/0414; G01N 27/44726; C12N 2310/20; C12N 2800/80; B01L 2300/021; B01L 3/505

(57) ABSTRACT

A kit includes reagents and materials for students to make gRNA and Target DNA, cut Target DNA with Cas9 guided by the gRNA, and visualize the uncut and cut the Target DNA on an agarose gel. The kit may include a shipping box that is able to withstand transport of the reagents and materials, reagents and materials needed to perform the desired curriculum, and the curriculum necessary for using the kit. Additional curriculum may be included with extension activities as desired.

### CROSS-REFERENCE TO RELATED APPLICATIONS

This application claims the benefit of U.S. Provisional Application No. 63/082,192 filed Sep. 23, 2020, and hereby incorporated by reference.

### BACKGROUND OF THE INVENTION

The present invention relates generally to clustered regularly interspaced short palindromic repeats (CRISPR) and CRISPR-associated protein 9 (Cas9) systems and in particular to an educational kit which allows students to practice CRISPR-Cas9 protocols.

CRISPR is a family of DNA sequences found within the genomes of prokaryotic organisms such as bacteria and archaea that are known to play a key role in the antiviral defense system of prokaryotes. The Cas9 is an enzyme that uses guide RNA (gRNA) to guide itself to the right part of the genome (e.g., the target sequence of DNA). It then binds to the target sequence and acts as a pair of ‘molecular scissors’ to cut the two strands of DNA at the designated location. When cut, the cell in question naturally tries to repair the damage, and this mechanism involving the DNA's repair machinery is what enables scientists to edit genes by removing, adding, or altering sections of the DNA sequence. As such, the CRIPSR/Cas9 technology has potential for a wide variety of applications in basic biological research as well as development of biotechnology products and treatment of disease.

CRISPR-Cas9 technology is rapidly becoming commonplace in genetics research laboratories to manipulate genes and genomes to investigate important biological questions. Despite its newfound mainstream presence in research, it has not yet become a standard offering in undergraduate biology courses. To date, CRISPR-Cas9 technology has been successfully integrated in some undergraduate lecture and laboratory curricula, course-based undergraduate research experiences (CUREs), undergraduate research projects, and intensive undergraduate-level workshops. However, these reports of CRISPR-Cas9 introductions into undergraduate curricula have been mostly published by highly experienced researchers who have well-established laboratories and research programs in molecular genetics, making accessibility of CRISPR-Cas9 in the undergraduate classroom a persistent issue. The challenges of efforts to include CRISPR-Cas9 in undergraduate biology curricula are likely due to instructors' inexperience with this new technology and the absence of a straightforward workflow for implementing these techniques in undergraduate biology classrooms at different levels.

It is therefore desired that students are taught CRISPR-Cas9 techniques so that they are better prepared for the workforce in this field.

### SUMMARY OF THE INVENTION

The present invention provides a kit including reagents and materials for students to make gRNA and Target DNA, cut the Target DNA with Cas9 guided by the gRNA, and visualize the uncut and cut Target DNA on an agarose gel. The kit may include a shipping box that is able to withstand transport of the reagents and materials, reagents and materials needed to perform the desired curriculum, and the curriculum necessary for utilizing the kit. Additional curriculum may be included with extension activities as desired.

It is thus a feature of at least one embodiment of the present invention to assess student learning in CRISPR-Cas9 technology and to research the mechanisms of human disease and discover potential therapeutics.

The present invention provides a CRISPR-Cas9 kit comprising a sealable package including: parafilm for mixing the loading dye with DNA thereon; 0.2 milliliter tubes; 1.5 milliliter tubes; screw-cap tubes; falcon conical centrifuge tubes; and reagents selected from a group consisting of: forward primer, reverse primer, gene specific oligo, tail oligo, PCR (master mix), T4 DNA polymerase, dNTPs, NEB buffer 2.1, T7 RNA polymerase, NTPs, buffer, DTT, RiboGuard RNase Inhibitor, DNase I, ammonia acetate, EtOH, RNA storage solution, Cas9, NEBuffer 3.1, proteinase K, agarose, TAE buffer, DNA gel stain, DNA ladder, loading dye, and nuclease-free water.

The kit may further comprise of at least one of pipette tips, pipettes stand, and a vortex mixer.

The kit may further comprise of at least one of tube labels, bag and box labels, and resealable bags.

The kit may further comprise of at least one of an insulated container and dry ice.

The present invention may provide a method of performing CRISPR-Cas9 techniques, comprising of providing a kit as described above and performing at least one of the following CRISPR-Cas9 techniques: annealing oligos and making the DNA Template, gRNA synthesis, DNase I treatment, and RNA precipitation, and in vitro nuclease assay with CRISPR-Cas9.

The technique of annealing oligos and making the DNA Template may comprise the following steps: in a PCR tube labeled with group number, “Annealed Oligos”, and date, combining the following:

| | |
|---|---|
| gene-specific oligo (100 μM): | 1.0 μL |
| tail oligo (100 μM): | 1.0 μL |
| nuclease-free water: | 8.0 μL; |

and
placing the PCR tube labeled “Annealed Oligos” in a thermocycler with the following program:
95° C. for 5 mins
Ramp down 0.1° C. per sec. to 25° C.
4° C. for infinity.

The technique of annealing oligos and making a DNA Template further comprises the following steps: transferring 5 μL of the annealed oligos to the PCR tube labeled DNA Template; transferring 5 μL of the Extension Master Mix (Ext. MM) to the PCR tube labeled DNA Template; wherein an “Annealed Oligos” tube can be stored at −20° C.; wherein the Extension Master Mix contains the following:

| | |
|---|---|
| dNTPs (10mM): | 1.25 μL per reaction; |
| NEB Buffer 2.1 (10X): | 1 μL per reaction; |
| T4 DNA polymerase: | 1 μL per reaction; |
| Nuclease-free water: | 1.75 μL per reaction; |

placing the PCR tube labeled DNA Template in the thermocycler with the following program:
12° C. for 45 mins. (DNA Template is made);
95° C. for 10 sec. (denatures T4 DNA polymerase, but oligos also denature);
Ramp down 0.1° C. per sec. to 25° C. (˜12 mins.; oligos anneal); 4° C. for infinity; and
   storing the tube labeled DNA Template tube at −20° C.

The technique of gRNA synthesis, DNase I treatment, and RNA precipitation may comprise the following steps: transferring 1.5 μL of DNA Template to the “gRNA w/DNA” tube; transferring 3.5 μL of gRNA MINI to the “gRNA w/DNA” tube wherein the gRNA MM contains the following:

| | |
|---|---|
| T7 RNA Polymerase: | 0.5 μL; |
| Transcription Reaction Buffer (10X): | 0.5 μL; |
| ATP (100 mM): | 0.5 μL; |
| GTP (100 mM): | 0.5 μL; |
| CTP (100 mM): | 0.5 μL; |
| UTP (100 mM): | 0.5 μL; |
| DTT (100 mM; helps inhibit RNases): | 0.5 μL; |
| RNase Inhibitor: | 0.12 μL; |

placing the “gRNA w/DNA” tube in a thermocycler with the following program:
37° C. for 30 min.;
   to the “gRNA w/DNA” tube, adding 15 μL of the DNase I MM (DI MM) for a total reaction volume of 20 μL; incubating at 37° C. for 30 min.; transferring 20 μL of gRNA from the “gRNA w/DNA” tube to the gRNA tube; to the gRNA tube, adding 70 μL of the RNA Prec. MM; pipetting up and down three times to mix; and incubating the gRNA tube at −20° C. for 30 min.

The technique of gRNA synthesis, DNase I treatment, and RNA precipitation may further comprise the following steps: centrifuging the gRNA tube 15 min. at 4° C. at 12,000 rpm; pouring out supernatant; transferring 1 mL of RNA Wash (70% EtOH) to the gRNA tube; centrifuging 5 min at 4° C. at 12,000 rpm; removing supernatant via pouring and using a P10 pipette with a tip; drying ˜15 min. at room temperature until pellet is clear; transferring 30 μL of RNA Storage Solution (1 mM sodium citrate, pH 6.5) to the gRNA tube; pipetting up and down five times to mix; and storing at −20° C.

The technique of in vitro nuclease assay with CRISPR-Cas9 may comprise the following steps: in the Target DNA tube, combining the following:
1 μL of DNA (1,000 ng/μL; from the zebrafish tail fin);
1 μL of Forward Primer (10 micromolar);
1 μL of Reverse Primer (10 micromolar);
22 μL of PCR MM;
   after PCR tubes have been prepared, placing the tubes in the designated thermal cycler set with the following program:
Cycle 1: Initial Denaturation: 95° C. for 3 minutes;
Cycle 2: Denaturation: 95° C. for 30 seconds, Annealing: 62° C. for 30 seconds, Extension: 72° C. for 1 minute, and repeating this cycle 35 times;
Cycle 3: Final Extension: 72° C. for 10 minutes;
Cycle 4: Hold at 4° C. for co;
   combining 6× Loading Dye+1 μL Target DNA on parafilm and adding to the appropriate lane in a gel; running the gel for 30 minutes at 120 V; and viewing the gel in a gel doc.

The technique of in vitro nuclease assay with CRISPR-Cas9 may further comprise the following steps: transferring 1 μL of gRNA to the “Cut Target DNA” tube; transferring 5 μL of the Cas9 MM to the “Cut Target DNA” tube, wherein the Cas9 MM includes the following:
Cas9: 0.6 μL;
NEBuffer 3.1 (10×): 1.0 μL;
Nuclease-free water: 3.4 μL;
   incubating the “Cut Target DNA” tube at 37° C. for 10 minutes; to the “Cut Target DNA” tube, adding 4 μL of the Target DNA; incubating the “Cut Target DNA” tube at 37° C. for 30 minutes; to the “Cut Target DNA” tube, adding 1 μL of Proteinase K (20 mg/mL); and incubating at 37° C. for 30 minutes.

The technique of in vitro nuclease assay with CRISPR-Cas9 further comprises the following steps: to a 250-mL Erlenmeyer flask adding the following:
50 mL 1×TAE buffer;
1 g agarose;
   microwaving the agarose for 1.5 minutes in a microwave and removing the flask from the microwave; adding 5 μL DNA gel stain to the agarose and swirling; pouring the agarose into the gel-casting tray; placing the 8-well comb into the gel-casting tray; once the gel has solidified, removing the comb from the solidified gel; placing the gel in an electrophoresis chamber with wells facing the negative electrode end; filling the chamber with 1×TAE to the max fill line; combining the following:
Lane 1: 2 μL ladder;
On parafilm: 1 μL Loading Dye+2 μL Target DNA;
To the “Cut Target DNA” tube, add 2 μL Loading Dye, pipet up and down 5 times to mix;
   running the gel at 120 V for 30 min; and viewing the gel with blue or UV light.

These particular objects and advantages may apply to only some embodiments falling within the claims and thus do not define the scope of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee. The disclosure will be better understood and features and aspects beyond those set forth above will become apparent when considering the following detailed description. The detailed description makes reference to the following figures.

FIG. 1 is a flowchart of a CRISPR-Cas9 kit according to one embodiment of the present invention showing the reagents of the CRISPR-Cas9 kit being stored in a freezer prior to shipping, shipment of the reagents and materials within an insulated container and a mailing box respectively, and shipping the CRISPR-Cas9 kit to a final destination where the CRISPR-Cas9 kit may be used by students.

FIG. 2 is a diagram of an overview of the CRISPR-Cas9 curriculum and correspondence with the flow of genetic information (Central Dogma of Molecular Biology). The yellow box shows the flow of genetic information culminating in the effect of gene function. The cream-colored boxes indicate bioinformatics. Boxes in dark blue indicate laboratory exercises, and boxes in light blue indicate variations and potential extensions for those lab exercises. CRISPR=clustered regularly interspaced short palindromic repeats; sgRNA=single guide RNA.

FIG. 3 is a diagram of laboratory activities associated with a sample CRISPR-Cas9 curriculum. Phase 1 of the workflow involves gene analysis using a genome browser (e.g., ENSEMBL) and CRISPR/sgRNA design using bioinformatics tools (e.g., Benchling, CRISPRScan, ChopChop). In Phase 2, a double-stranded DNA (dsDNA) that will be used as a template for sgRNA synthesis is prepared. Phase 3 involves the synthesis of sgRNA by T7 RNA polymerase-driven in vitro transcription followed by quantitative and qualitative analysis of the synthesized RNA. During Phase 4, the nuclease activity of the sgRNA-Cas9 ribonucleoprotein complex on the PCR-amplified genomic target region is tested in vitro. CRISPR=clustered regularly interspaced short palindromic repeats; sgRNA=single guide RNA.

FIG. 4 are sample gels prepared and analyzed by students demonstrating in vitro nuclease assay of CRISPR-Cas9. The gel image in the left panel shows the efficacy of various sgRNA guides (C1-C4), along with various experimental controls to demonstrate the conditions required for optimal nuclease activity. The gel in the right panel shows nuclease activity at different concentrations of the sgRNA guide. CRISPR=clustered regularly interspaced short palindromic repeats; sgRNA=single guide RNA.

FIG. 5 is an exemplary implementation plan of the CRISPR-Cas9 curriculum in a student laboratory. sgRNA=single guide RNA; RT-PCR=reverse transcriptase PCR.

FIG. 6 is a diagram of an alternative embodiment of laboratory activities associated with a sample CRISPR-Cas9 curriculum.

FIG. 7 is a diagram of Part A of the laboratory activities of FIG. 6 associated with making the DNA Template.

FIG. 8 is a diagram of Part B1 of the laboratory activities of FIG. 6 associated with making the gRNA.

FIG. 9 is a diagram of Part B2 of the laboratory activities of FIG. 6 associated with making the gRNA.

FIG. 10 is a diagram of Part C of the laboratory activities of FIG. 6 associated with making the target DNA.

FIG. 11 is a diagram of Part D of the laboratory activities of FIG. 6 associated with cutting the target DNA with Cas9/gRNA.

FIG. 12 is a diagram of Part E of the laboratory activities of FIG. 6 associated with visualizing the uncut/cut target DNA on an agarose gel.

FIG. 13 is a sample gel prepared and analyzed by students demonstrating in vitro nuclease assay of CRISPR-Cas9.

FIG. 14 is a reference image of a DNA ladder with easy to identify reference bands used by students to indicate where the band should appear in the “Expected” lane during the hypothesis step.

FIG. 15 is a diagram used with respect to the Annealing, Extending, and Transcribing activity.

FIG. 16 is a diagram used with respect to the Annealing, Extending, and Transcribing activity.

FIG. 17 is an annotated calmla genomic sequence used with respect to the Calculating the Size of the calmla Target DNA and Cut Target DNA Products activity.

FIG. 18 is the zebrafish calmla mRNA from start codon to stop codon, zebrafish calm1a amino acid sequence, and human calm1 amino acid sequence used with respect to the Comparing Zebrafish and Human CALM1 Amino Acid Sequences activity.

FIG. 19 is a diagram used with respect to the In Vitro Nuclease Assay activity.

FIG. 20 is a diagram used with respect to the In Vitro Nuclease Assay activity.

FIG. 21 is a diagram used with respect to the In Vitro Nuclease Assay activity.

FIG. 22 is a diagram used with respect to the In Vitro Nuclease Assay activity.

FIG. 23 is a diagram used with respect to the In Vitro Nuclease Assay activity.

FIG. 24 is a sample gel of the DNA ladder and the uncut and cut calmla Target DNA.

### DETAILED DESCRIPTION OF THE PREFERRED EMBODIMENT

Storage and Shipping

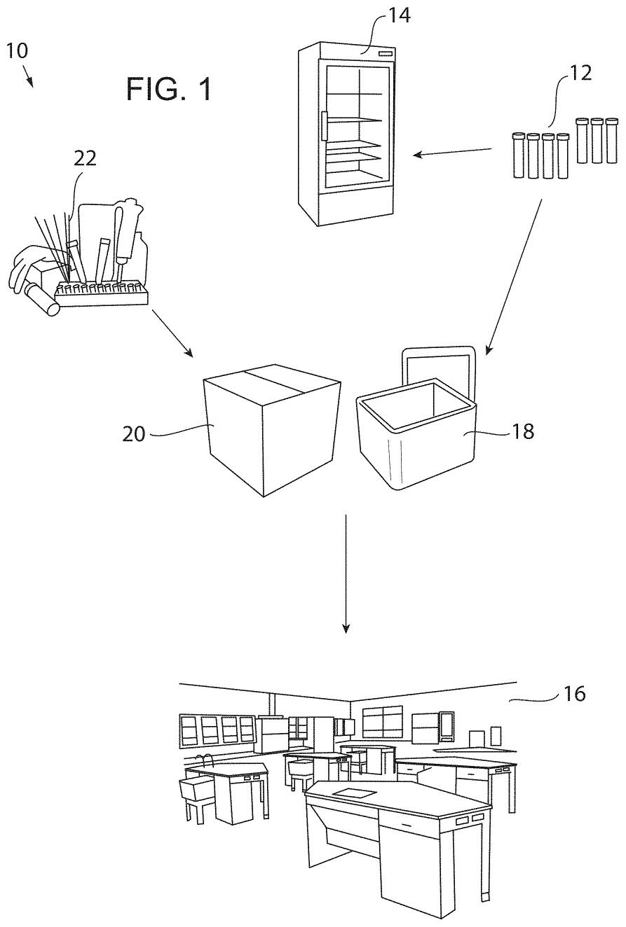

Referring to FIG. 1, prior to the delivery of the CRISPR-Cas9 kit 10, kit reagents 12, as further described below, may be stored within a freezer 14. The freezer 14 may maintain the kit reagents 12 at an approximately −20° C. storage temperature before being shipped to the student environment 16.

The CRISPR-Cas9 kit 10 may be shipped within an insulated container 18 holding the kit reagents 12 and may further contain dry ice with the insulated container 18 labeled with a “dry ice” label. A mailing box 20 may contain kit materials 22, as further described below, which do not need to be kept cold before, during, or after shipment. The insulated container 18 and mailing box 20 may be sealed with shipping tape and may further include packing list envelopes with invoices.

Kit Materials

In one embodiment of the present invention, the CRISPR-Cas9 kit 10 may include the following kit materials 22:
parafilm for mixing the loading dye with DNA thereon
0.2 milliliter tubes
1.5 milliliter tubes
screw-cap tubes
falcon conical centrifuge tubes
tube labels
bag and box labels
resealable bags (to contain the falcon tubes and storage box)

The following kit materials 22 may be included in the CRISPR-Cas9 kit 10, or alternatively, may be materials or equipment found in a basic laboratory which are not included as part of the CRISPR-Cas9 kit 10.

These kit materials may optionally include P1000, P200, P20, and P2 pipettes for dispensing the required volumes of reagents into the tubes. A pipettes stand may be used to keep the pipettes organized. Pipette tips to fit the various sizes of pipettes may be included with the kit materials 22.

A vortex mixer may ensure the reagents are mixed prior to aliquoting and to ensure proper concentration of the reagents. After mixing the reagents with the vortex mixer, reagents may be briefly spun down using a mini centrifuge and its platform.

It is understood that the CRISPR-Cas9 kit 10 may include enough kit materials 22 to allow at least eight students or groups of students to individually perform the curriculum described below.

Kit Reagents

In one embodiment of the present invention, the CRISPR-Cas9 kit 10 may include several kit reagents 12, for example, as listed below. Additional oligos may be included which are specific for different genes.

calm 1a_FP:
(SEQ ID NO: 1)
TACCGCTGGATGCCAACCGAGA
calm 1a_RP:
(SEQ ID NO: 2)
GCAGCTGAGGCCTGATGCTCAG
calm 1a_GSO:
(SEQ ID NO: 3)
taatacgactcactataggAAAATTAATACGCAACCTTCgttttagagct
agaa
Tail Oligo:
(SEQ ID NO: 4)
AAAAGCACCGACTCGGTGCCACTTTTTCAAGTTGATAACGGACTAGCC
TTATTTTAACTTGCTATTTCTAGCTCTAAAAC
(Eurofins Genomics)

dNTP Mix (10 mM each) (Thermo Fisher Scientific R0191)
NEB Buffer 2.1 (New England Biolabs B7202S)
T4 NEB DNA Polymerase (New England Biolabs M0203S)
AmpliScribe T7-Flash Kit (Lucigen ASF3257)
DNase I, RNase-free (Thermo Fisher Scientific EN0521)
RNA Storage Solution
NEB Buffer 3.1
Alt-R® S.p. Cas9 Nuclease V3, 100 μg (Integrated DNA Technologies (IDT) 1081058)
EnGen® Spy Cas9 NLS (note: two NLS) (New England Biolabs M0646T)
Diluent B (note: for in vitro reaction with EnGen® Spy Cas9 NLS) (New England Biolabs B8002S)
agarose for gels
Nuclease-free water
DNA ladder
DNA Gel Stain and other related agarose gel reagents

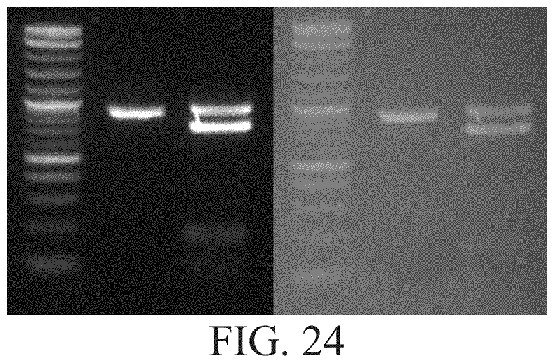

The reagents 12 may be associated with the following general curriculum steps:
1. Oligonucleotides for making the DNA Template (gene specific oligo and tail oligo) and PCR to make the Target DNA (forward primer and reverse primer)
2. Extension of oligos to form the DNA Template (T4 DNA polymerase, dNTPs, NEB buffer 2.1)
3. Form the gRNA (T7 RNA polymerase, NTPs, buffer, DTT, RiboGuard RNase Inhibitor)
4. Remove the DNA Template (DNase I)
5. Precipitate and wash the gRNA (ammonia acetate and EtOH)
6. Store the RNA (RNA storage solution)
7. PCR (master mix)
8. Perform the in vitro nucleus reaction in which the gRNA guides the Cas9 to the Target DNA to cut the DNA (Cas9, NEBuffer 3.1, proteinase K)
9. Run an agarose gel to visualize the uncut and cut Target DNA (agarose, TAE buffer, DNA gel stain, DNA ladder, loading dye) (see ladder of the uncut and cut calm1a target DNA FIG. 24).
10. Nuclease-free water for the reactions to take place in

It is understood that the CRISPR-Cas9 kit 10 may include enough reagents 12 to allow at least eight students or groups of students to individually perform the curriculum described below.

Kit Curriculum

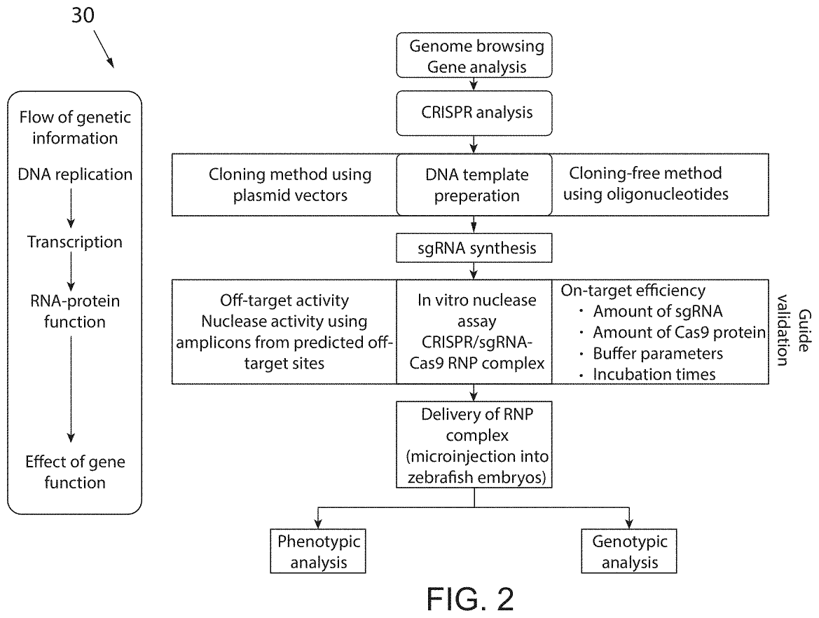

Referring to FIG. 2, the curriculum overview 30 may be designed to implement effective genome editing tools in student classrooms, with particular attention to the limited scientific resources of these environments. Interspersed with laboratory activities, discussions on pedagogical issues and classroom activities are also conducted within the curriculum.

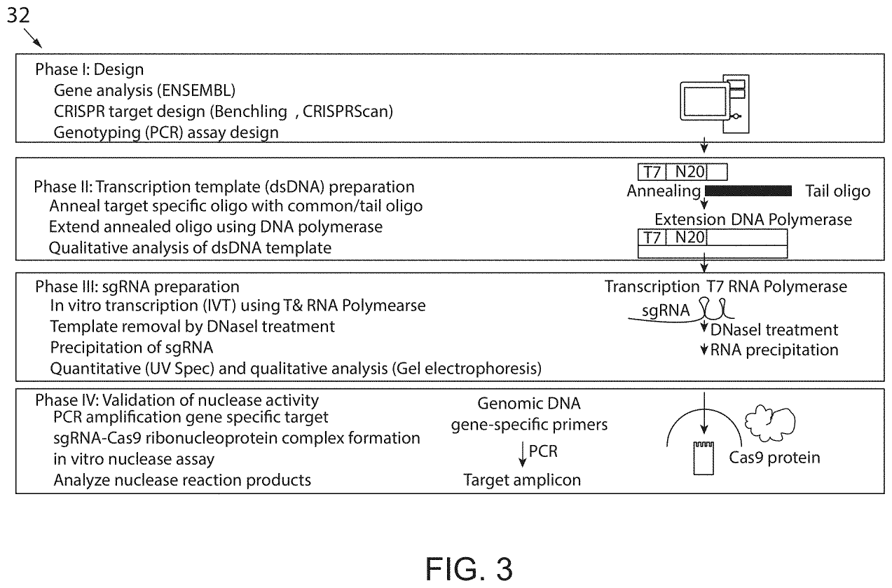

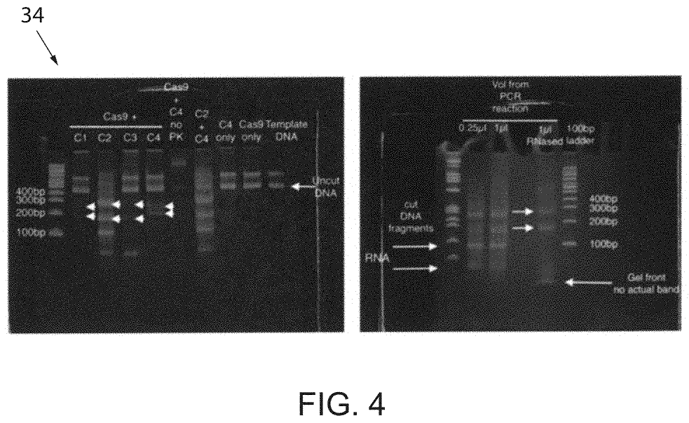

Referring to FIGS. 3 and 4, the laboratory steps of the sample curriculum 32 may be for use with the zebrafish (Danio rerio), however, the curriculum shown in FIG. 3 can be implemented in vitro and extended to be used in a variety of model organisms. The sample curriculum 32 enables students to select a gene of interest, design an appropriate single guide RNA (sgRNA) to cut a specific gene target via Cas9, and use in vitro techniques to investigate whether the design worked. The techniques outlined in FIG. 3 include in vitro transcription and nuclease assays 34 to illustrate a simple way in which students could test whether CRISPR-induced double strand breaks (DSBs) in the DNA they targeted were successful (see FIG. 4).

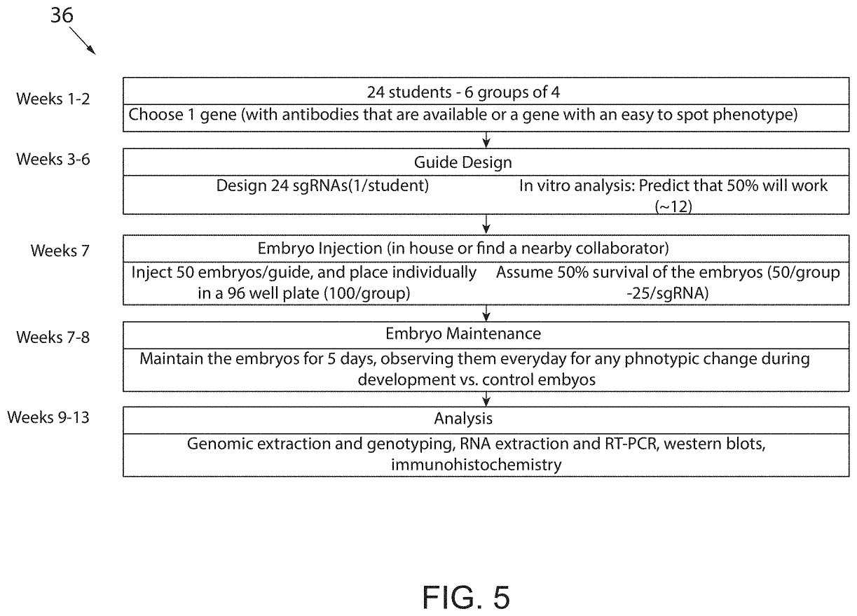

Referring to FIG. 5, the sample curriculum 32 may be implemented in courses at varying levels and engaging students at different stages of their training. The modular nature of the workflow allows the instructor to tailor it to the needs of a particular class or group of students (a sample implementation plan 36 devised by participants is shown in FIG. 5). An instructor may elect to use select portions of the workflow in order to leave time for other activities/projects that are important for a particular group of students. Also, an instructor can choose to emphasize bioinformatics, molecular biology, principles of cell and developmental biology, or genetic applications, depending on the needs of a particular class.

The CRISPR-Cas9 kit 10 may include a sufficient amount of the reagents 12 and materials 20 needed to perform the sample curriculum 32 which includes the following exemplary lesson plans. Example 1 below is optional and may be performed by the students or may be performed “in house” prior to delivery to the students. Examples 2 through 6 below may be performed by students as part of the sample curriculum 32. Examples 7 through 12 below are additional activities that may be performed by students as part of the sample curriculum 32.

### (Optional) Example 1: Resuspending and Diluting the Oligos

Purpose:

1. Resuspend oligos for a stock solution of 100 μM.

2. Make 10-μM working solutions.

Procedure:

1. Confirm that the oligo sequences on the labels are the same as the ones ordered.

2. Resuspend the oligos to make a 100-μM stock solution.
a. Centrifuge the oligos for 30 sec. at 12,000 rpm.
b. Resuspend each oligo with nuclease-free H₂O to a concentration of 100 μM. To determine the amount of water to add, multiple the number of nmol by 10. For example, if there are 25.0 nmol of oligo in the tube, add 250 μL nuclease-free H₂O.
c. Vortex 5 seconds.
d. Centrifuge briefly.
e. Label the tubes: 100 μM and today's date. Store at −20° C.

3. Make a 10-μM working solution for each oligo.
a. Label a 1.5-mL sterile tube with your group number, oligo name, 10 μM, today's date.
b. Add 90 μL nuclease-free H₂O to the new tube.
c. Transfer 10 μL of the 100-μM oligo solution.
d. Vortex 5 seconds.
e. Briefly centrifuge. Store at 4° C.

Results and Conclusion:

Summarize what was done and what the oligo solutions will be used for.

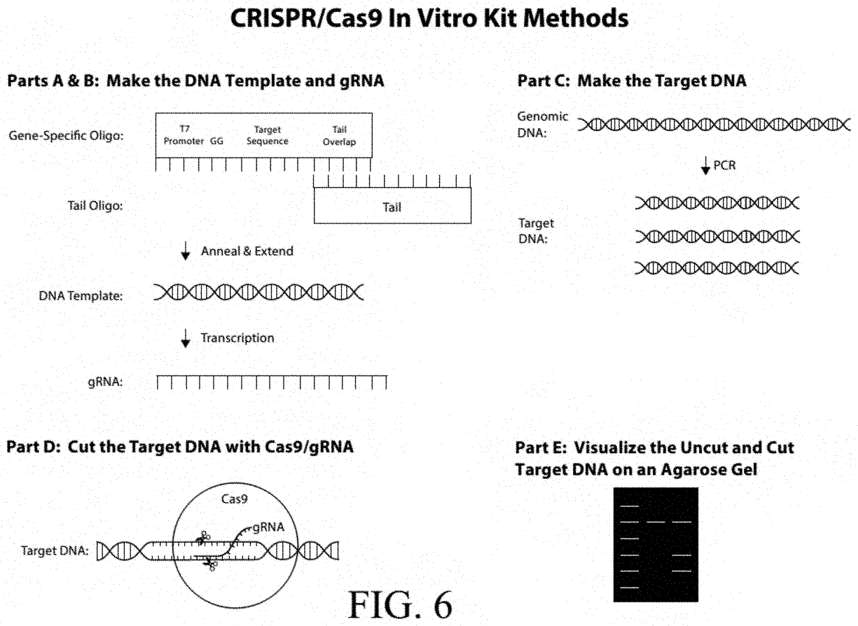

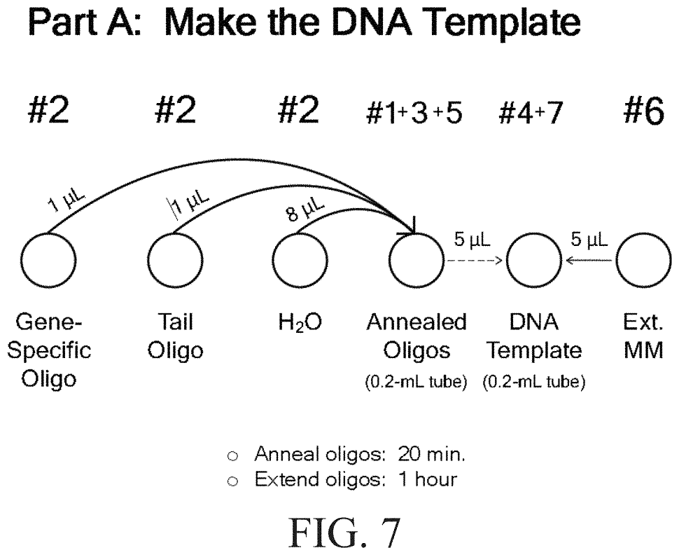

### Example 2: Part A: Make the DNA Template that Encodes gRNA (See FIGS. 6 and 7

Purpose:

1. Anneal (bind) the gene-specific oligo and tail oligo together.

2. Use T4 polymerase to extend the oligos to form the double-stranded DNA. template.

Hypothesis:

1. The gene-specific oligo and tail oligo should anneal together.

2. Double-stranded DNA should be made that will be used as the template to make gRNA.

Procedure:

Annealing the Oligos

1. Label a 0.2-mL tube with the following: “Annealed Oligos”, your group number, and today's date.

2. To your “Annealed Oligos” tube, combine the following. Pipet up and down 5 times to mix.

| | |
|---|---|
| gene-specific oligo (100 μM): | 1.0 μL |
| tail oligo (100 μM): | 1.0 μL |
| nuclease-free water: | 8.0 μL |

3. Place the “Annealed Oligos” tube in the thermocycler with the following program:
95° C. for 5 min. (oligos denature/become single-stranded)
Ramp down 0.1° C. per sec. to 25° C. (˜12 min. or turn off thermocycler for 15 min.; oligos anneal)
4° C. for infinity

Extending the Oligos: Create the DNA Template for Making the gRNA

4. Label a 0.2-mL tube with the following: DNA Template, your group number, and today's date.

5. To your “DNA Template” tube, add 5 μL of the annealed oligos.

6. Then, to the annealed oligos in your “DNA Template” tube, add 5 μL of the Extension Master Mix (Ext. MM). ONLY add the Extension Master Mix to the annealed oligos. DO NOT pipet up and down. The “Annealed Oligos” tube can be stored at −20° C.

The Extension Master Mix contains the following:

| dNTPs (10 mM): | 1.25 μL per reaction (becomes part of the |
|---|---|
| extended DNA) | |
| NEB Buffer 2.1 (10X): | 1 μL per reaction (provides the correct pH |
| for the reaction and magnesium for attaching the nucleotides) | |
| T4 DNA polymerase: | 1 μL per reaction (adds the nucleotides to |
| extend the DNA) | |
| nuclease-free water: | 1.75 μL per reaction (allows for the correct |
| concentration) | |

7. Place the “DNA Template” tube in the thermocycler with the following program.
12° C. for 45 mins. (The gRNA is made.)
95° C. for 10 sec. (denatures T4 DNA polymerase, but oligos also denature)
Ramp down 0.1° C. per sec. to 25° C. (˜12 mins. or turn off thermocycler for 15 min.; oligos anneal)
4° C. for infinity

8. Store the “DNA Template” tube at −20° C.

Results and Conclusion:

Make a figure showing annealing of the oligos and extension to form the DNA template.

Label the following parts on your diagram: T7 Promoter, GG, Gene-Specific Region, Tail Overlap, Tail.

Explain the purpose of each of the labeled parts.

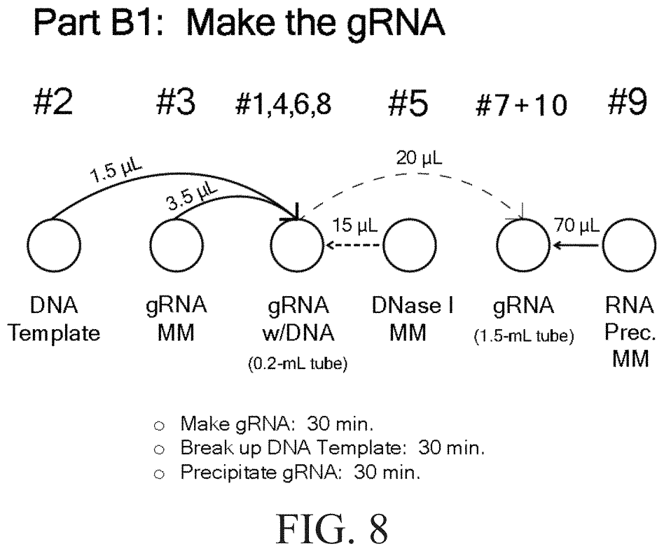

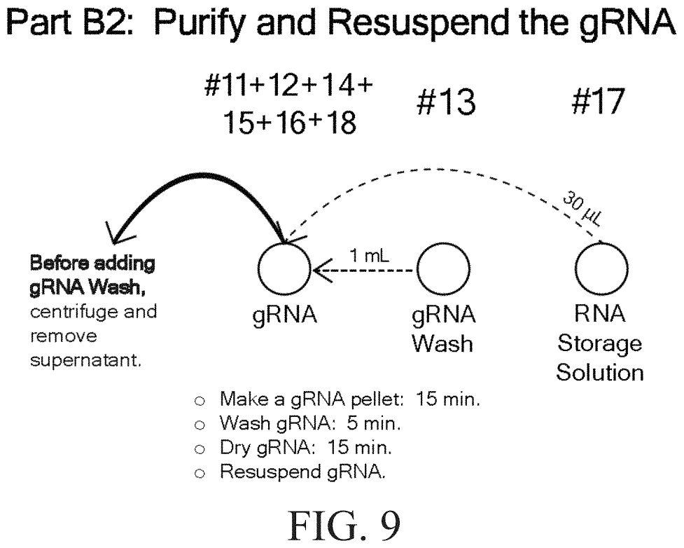

### Example 3: Part B: Make and Purify the gRNA (See FIGS. 6 and 8-9)

Purpose:

1. Make and purify gRNA that will guide Cas9 to the DNA Target.

Hypothesis:

1. A white RNA pellet should be observed after the precipitation step.

Procedure:

Part B1: Make the gRNA

Make the gRNA

1. Label a 0.2-mL tube with the following: gRNA w/DNA, your group number, and today's date.

2. To your “gRNA w/DNA” tube, add 1.5 μL of DNA Template.

3. To the “gRNA w/DNA” tube, add 3.5 μL of gRNA MM. Pipet up and down 5 times to mix. The gRNA MM contains the following:

| | |
|---|---|
| T7 RNA Polymerase: | 0.5 μL |
| Transcription Reaction Buffer (10X): | 0.5 μL |
| ATP (100 mM): | 0.5 μL |
| GTP (100 mM): | 0.5 μL |
| CTP (100 mM): | 0.5 μL |
| UTP (100 mM): | 0.5 μL |
| DTT (100 mM; helps inhibit RNases): | 0.5 μL |
| RNase Inhibitor: | 0.12 μL |

4. Place the “gRNA w/DNA” tube in the thermocycler with the following program:
37° C. for 30 min. (The gRNA is made.)

DNase I Treatment (Getting Rid of the DNA Template)

5. To the “gRNA w/DNA” tube, add 15 μL of DNase I MM (contains 0.5 μL of DNase I and 14.5 μL nuclease-free water) for a total reaction volume of 20 μL.

6. Place the “gRNA w/DNA” tube in the thermocycler with the following program:
37° C. for 30 min. (DNase I breaks apart the DNA Template.)

Precipitate the gRNA

7. Label a 1.5-mL tube with the following: gRNA, your group number, and today's date.

8. To the “gRNA” tube, add 20 μL of gRNA from the “gRNA w/DNA” tube.

9. Then, to the “gRNA” tube, add 70 μL of the RNA Prec. MM (contains 10 mL 5 M ammonium acetate and 60 μL 100% EtOH). Pipet up and down 5 times to mix.

10. Incubate the “gRNA” tube at −20° C. for 30 min. (The salt and ethanol precipitate the gRNA into a solid.) Also, place the RNA Wash at −20° C. (if not already at −20° C.) and start the refrigerated centrifuge cooling to 4° C.

Part B2: Purify and Resuspend the gRNA

11. Centrifuge the gRNA tube 15 min. at 4° C. at 12,000 rpm.

12. Without disturbing the cloudy white pellet, remove and discard the supernatant (liquid portion) with a P200 set to 90 μL.

13. Transfer 1 mL of the RNA Wash (70% EtOH) to the gRNA tube.

14. Centrifuge 5 min at 4° C. at 12,000 rpm.

15. Without disturbing the cloudy white pellet, remove and discard the supernatant with a P1000 and P10 set at maximum volume.

16. Place the tube horizontally on the counter with its lid open for 15 min. or until the pellet is clear. (gRNA Wash evaporates away.)

17. To the “gRNA” tube, add 30 μL of RNA Storage Solution (1 mM sodium citrate, pH 6.5). Pipet up and down 5 times to mix.

18. Store the “gRNA” tube at −20° C.

Results:

Draw a picture of the “gRNA” tube with its cloudy white pellet of gRNA.

Conclusion:

Summarize the process of transcription, including the purposes of the reagents.

State what the gRNA will be used for in the future.

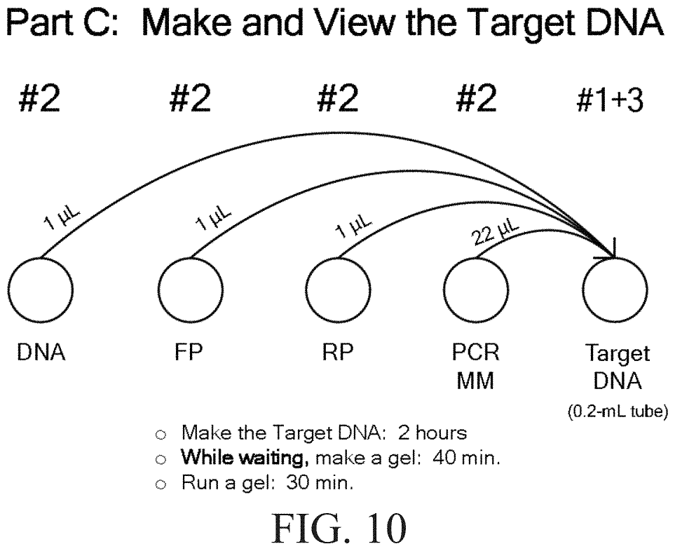

### Example 4: Part C: Make Copies of and View the Target DNA (see FIGS. 6 and 10)

Purpose:

1. Make copies of the Target DNA that will be cut in the future with Cas9.

2. View the Target DNA on an agarose gel to confirm the Target DNA was obtained.

Hypothesis:

1. A ______-bp product should be made. (Fill in the blank)

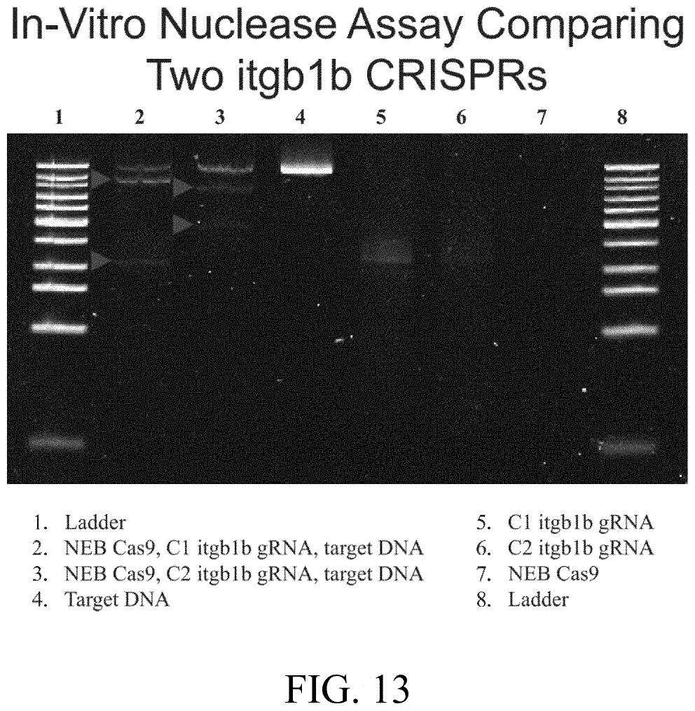

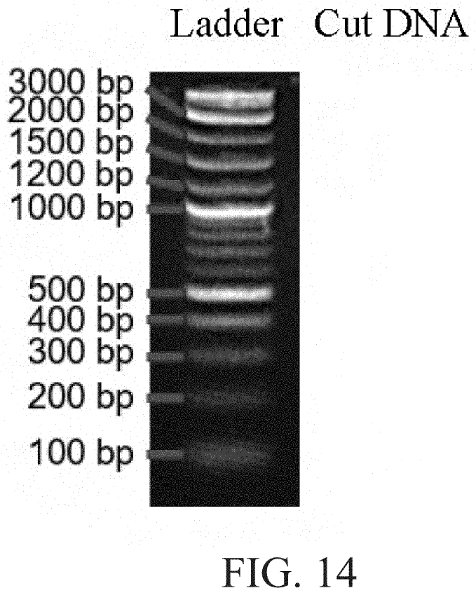

2. Draw where the Target DNA band should appear in the “Expected” lane. (see FIG. 14).

Procedure

1. Label a 0.2-mL tube with the following: Target DNA, your group number, and today's date.

2. In the “Target DNA” tube, combine the following. Pipet up and down 5 times to mix.
1 μL of the DNA (1,000 ng/μL; from the zebrafish tail fin)
1 μL of the Forward Primer (10 micromolar)
1 μL of the Reverse Primer (10 micromolar)
22 μL of the PCR MM (see note below)

Note: the PCR Master Mix contains buffer (keeps pH appropriate for the reaction), 0.4 mM dNTPs (building blocks of the PCR product), Taq polymerase (a protein that makes the PCR product), 4 mM MgCl₂ (allows Taq polymerase to bind the dNTPs), and Nuclease-Free H₂O (for the appropriate concentration)

3. After all the groups' PCR tubes have been prepared, place your tube in the thermocycler set with the following program:
Cycle 1: Initial Denaturation: 95° C. for 3 minutes (activates polymerase; denatures DNA)
Cycle 2: Denaturation: 95° C. for 30 seconds (denatures DNA to separate the DNA strands)
Annealing: 62° C. for 30 sec. (primers bind/anneal DNA; note: the temperature depends on the primers and DNA polymerase used; when using Taq polymerase, average the melting temperatures of the primers and subtract 5° C.)
Extension: 72° C. for 1 minute (for a product size 2,000 bp or less) (DNA polymerase makes/synthesizes DNA.)
Repeat this cycle 35 times (amplifies product to detectable level)
Cycle 3: Final Extension: 72° C. for 10 minutes (completes PCR products to full length)
Cycle 4: 4° C. for co (store the DNA short-term)

4. During the wait, watch how the instructor makes 2% agarose gels for the class:
50 mL 1×TAE+1 g agarose microwaved for 1.5 min. (three 30-sec. increments, swirling in between) in a 250-mL Erlenmeyer flask. Caution: the flask and its contents will become very hot! Use hot mitts or a folded paper towel to handle flask.
Let flask cool 20 sec.
Add 5 μL DNA gel stain (binds DNA for visualizing in blue or UV light), gently swirling to mix.
Pour the flask contents into the gel-casting tray. Note: depending on the size tray, one to two gels can be made.
Insert 8-well (or 8+ well) comb.
Let gel solidify 40 min.
Remove comb, and transfer gel into the electrophoresis chamber.
Cover gel with lx TAE.

5. Pipet 2 μL of DNA Ladder into your designated well in the gel.

6. Combine 1 μL 6× Loading Dye+2 μL Target DNA on parafilm before loading the 3 μL into your next designated well in the gel.

7. Run the gel for 30 min. at 120 V.

8. Observe the gel with blue light or UV light.

Results:

Attach or draw a picture of the gel. Label the lanes of the gel and the sizes of the brands.

Conclusion:

State what size Target DNA product was obtained.

Explain what the Target DNA will be used for in the future.

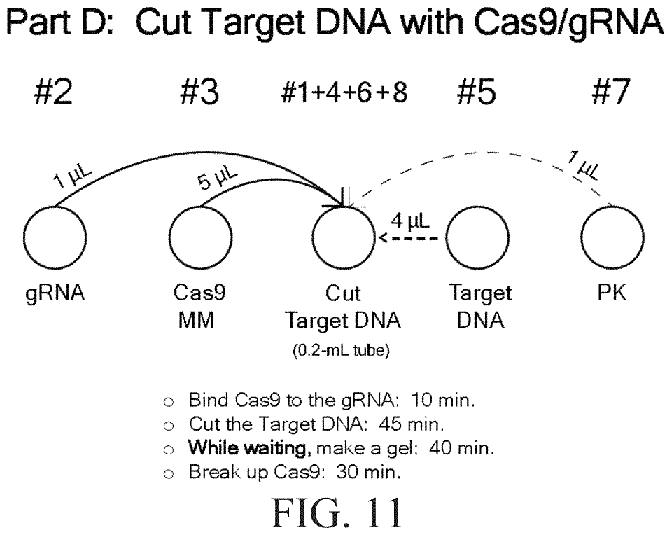

### Example 5: Part D: Cut the Target DNA with Cas9/gRNA (see FIGS. 6 and 11)

Purpose:

1. Test whether the gRNA can guide Cas9 to the Target DNA to allow Cas9 to cut the Target DNA.

Hypothesis:

1. The gRNA should bind Cas9 and the Target DNA, allowing Cas9 to cut the Target DNA.

Procedure:

Bind Cas9 to the gRNA

1. Label a 0.2-mL tube with the following: Cut Target DNA, your group number, and today's date.

2. To the “Cut Target DNA” tube, add 1 μL of gRNA. (The gRNA will guide the Cas9 to the correct location to cut the DNA.)

3. Then, to the “Cut Target DNA” tube, add 5 μL of the Cas9 MM. Pipet up and down 5 times to mix. The Cas9 MM includes the following:

| | |
|---|---|
| Cas9: | 0.6 μL |
| NEBuffer 3.1 (10X): | 1.0 μL |
| nuclease-free water: | 3.4 μL |

4. Place the “Cut Target DNA” tube in the thermocycler with the following program:
37° C. for 10 min. (Cas9 binds the gRNA.)

Bind Cas9/gRNA to the Target DNA

5. To the “Cut Target DNA” tube, add 4 μL of the Target DNA. ONLY add the Target DNA to the Cas9/gRNA. DO NOT pipet up and down.

6. Place the “Cut Target DNA” tube in the thermocycler with the following program:
37° C. for 30 min. (The gRNA binds the Target DNA, allowing Cas9 to cut the Target DNA.)

Break Up Cas9 with Proteinase

7. To the “Cut Target DNA” PCR tube, add 1 μL of Proteinase K (20 mg/mL) (breaks apart Cas9). Pipet up and down 5 times to mix.

8. Place the “Cut Target DNA” tube in the thermocycler with the following program:
37° C. for 30 min. (Proteinase K breaks apart Cas9, freeing the gRNA and DNA.)

Results and Conclusion

Summarize what occurs during the following: 1) incubation of Cas9 with gRNA, 2) incubation of Cas9/gRNA with the Target DNA, and 3) the proteinase treatment.

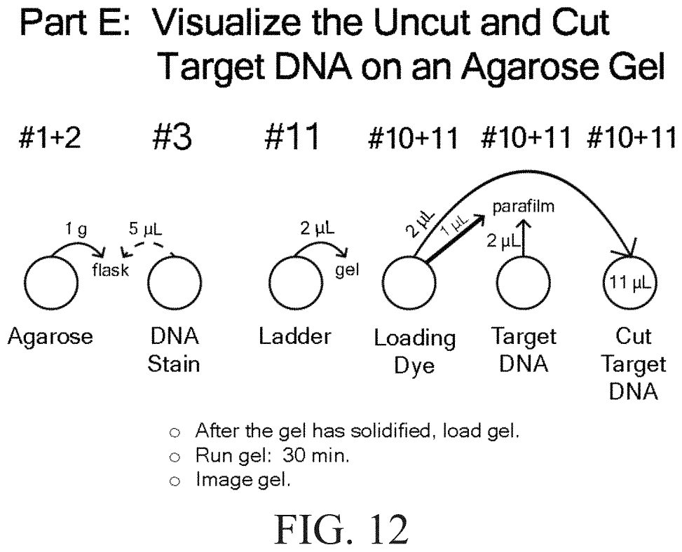

### Example 6: Part E: Visualize the Uncut and Cut Target DNA on an Agarose Gel (see FIG. 6 and FIGS. 12-13)

Purpose:

1. Separate and visualize the products of the Cut Target DNA on an agarose gel to provide evidence that 1) the gRNA was able to guide the Cas9 to the Target DNA and 2) Cas9 cut the Target DNA.

Hypothesis:

1. Cas9 should cut the Target DNA, resulting in two DNA products (bp and bp), as shown in FIG. 14. (Fill in the blanks)

Safety:
Wear gloves and eye protection.
Use the folded paper towel method (as demonstrated by instructor) or oven mitts when handling the Erlenmeyer flask; the agarose will be heated to boiling.
Use dry gloves when operating the power supply.
Limit your exposure to UV if using UV.

Procedure:

1. To a 250-mL Erlenmeyer flask, add the following:
50 mL 1×TAE buffer (contains ions that make the current that moves the negatively-charged DNA)
1 g agarose (creates a mesh that separates DNA based on size)

2. Microwave the agarose for 1.5 min., swirling every 30 sec. (dissolves the agarose). Caution: the flask and its contents will become very hot! Use hot mitts or a folded paper towel to handle flask.

3. After 20 sec., add 5 μL DNA gel stain to the agarose (binds DNA for visualizing in blue or UV light). Use hot mitts or a folded paper towel to gently swirl to mix.

4. Pour the flask's contents into the gel-casting tray. Note: depending on the size tray, one to two gels can be made.

5. Place the 8-well (or 8+ well) comb into the gel-casting tray.

6. Remove any bubbles with a P10 pipette tip.

7. After 40 min. (once the gel has solidified), remove the comb.

8. Place the gel in the electrophoresis chamber with the wells facing the negative electrode end. (The negative electrodes will repel the negatively-charged DNA, allowing the DNA to migrate across the gel.)

9. Cover the gel with 1×TAE.

10. Combine the following:
On parafilm: 1 μL Loading Dye+2 μL Target DNA
To the “Cut Target DNA” tube, add 2 μL Loading Dye; pipet up and down 5 times to mix.

11. Load the gel with the following:
Lane 1:2 μL ladder
Lane 2: 3 μL Target DNA with Loading Dye
Lane 3: 13 μL Cut Target DNA with Loading Dye

12. Run the gel for 30 min. at 120 V.

13. Observe the gel with blue or UV light.

Results:

Attach or draw a picture of the gel. Label the lanes of the gel and the sizes of the bands.

Conclusion:

Explain whether the results of the gel provide evidence that Cas9 was guided by the gRNA and cut the Target DNA.

Look back at the past protocols and describe three changes that could result in more cut Target DNA.

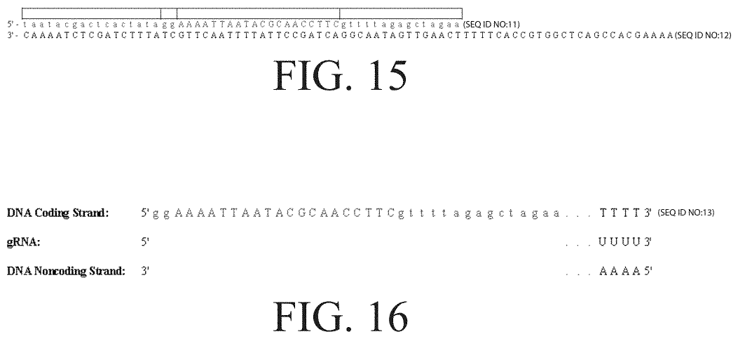

### Example 7: Annealing, Extending, and Transcribing Activity (FIGS. 15-16)

Gene-Specific Oligo:
(SEQ ID NO: 5)
5′-taatacgactcactataggAAAATTAATACGCAACCTTCgttttaga
gctagaa-3′
Tail Oligo:
(SEQ ID NO: 6)
5′-AAAAGCACCGACTCGGTGCCACTTTTTCAAGTTGATAACGGACTAGC
CTTATTTTAACTTGCTATTTCTAGCTCTAAAAC-3′
Reverse Sequence of Tail Oligo:
(SEQ ID NO: 7)
5′-CAAAATCTCGATCTTTATCGTTCAATTTTATTCCGATCAGGCAATAG
TTGAACTTTTTCACCGTGGCTCAGCCACGAAAA-3′

Source: https://www.bioinformatics.org/sms/rev_comp.html->Reverse

1. Anneal the oligos below by cutting and pasting the Tail Oligo (in black letters) such that its 5′ nucleotides complementary base pair with the nucleotides on the 3′ end of the Gene-Specific Oligo. See FIG. 15.

Hint: CAAAA binds gtttt in the Gene-Specific Oligo.

2. Extend the oligos by adding the correct nucleotides (A-T and G-C).

For the left side, determine which bases complementary base pair with the Gene-Specific Oligo.

For the right side, copy the nucleotides remaining in the Tail Oligo, paste the sequence at https://www.bioinformatics.org/sms/rev_comp.html, select complement, and click SUBMIT.

3. Count the number of base pairs in the DNA Template that you just created in FIG. 15.

Length of DNA Template (in bp):

4. The first part of the DNA Template contains the T7 promoter (taatacgactcactata), which T7 RNA polymerase binds to.

Label the box above the DNA Template that corresponds to the T7 Promoter: T7 Promoter.

5. T7 RNA polymerase binds to its promoter better if the promoter sequence is followed by two guanines (gg). Label the two guanines in FIG. 15: GG.

Of note, the GG can be added if the Target Sequence doesn't start with GG. If the Target Sequence starts with one G, one G is added after the T7 promoter for a total of two guanines.

6. The Target Sequence contains around 20 base pairs that are specific to the location where Cas9 should cut the DNA.

The Target Sequence can be found in the same strand of DNA as the gene (coding, sense, or nontemplate strand) or the opposite strand (noncoding, antisense, or template strand).

If the Target Sequence is in the coding strand, the Target Sequence in the future gRNA will bind the complementary bases in the noncoding strand.

If the Target Sequence is in the noncoding strand, the Target Sequence in the future gRNA will bind the reverse complementary sequence in the coding strand.

Label the Target Sequence in FIG. 15.

7. The last part of the Gene-Specific Oligo contains bases complementary to the Tail Oligo and is called the Tail Overlap. Label the Tail Overlap in FIG. 15.

Of note, the tail part of the gRNA binds to Cas9.

8. After the T7 RNA polymerase binds its promoter, it starts transcribing and making the gRNA by attaching the correct ribonucleotides (A, U, G, C).

Copy the DNA noncoding strand that you created in FIG. 15 (starting with CCTT), and paste it in the “DNA Noncoding Strand” row in FIG. 16.

Use the DNA Noncoding Strand to transcribe the gRNA. Notice that the gRNA looks like the DNA Coding Strand, except it has U's in place of the T's. See FIG. 16.

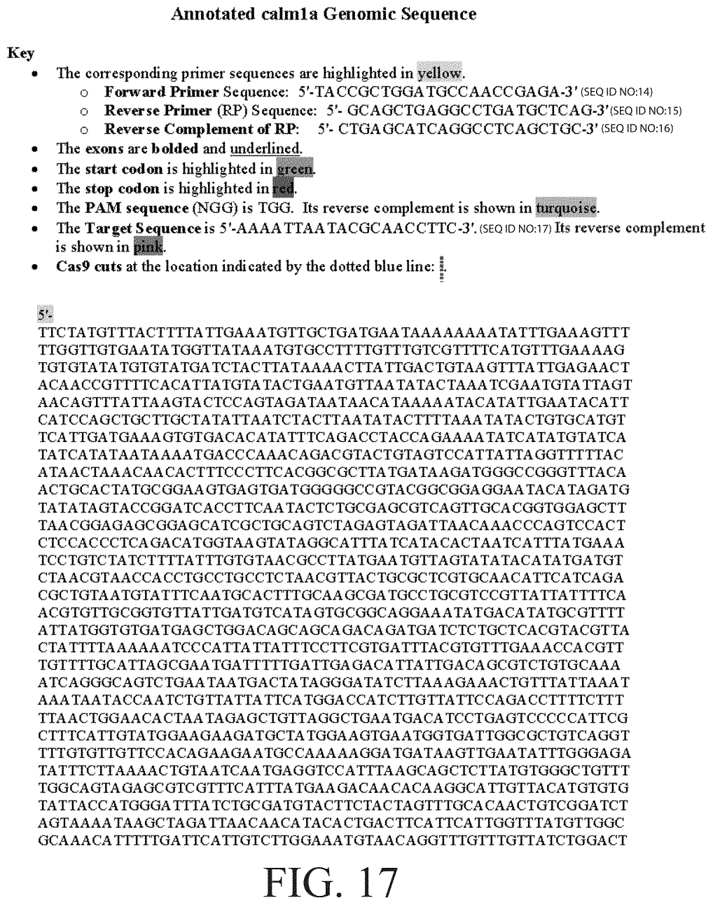

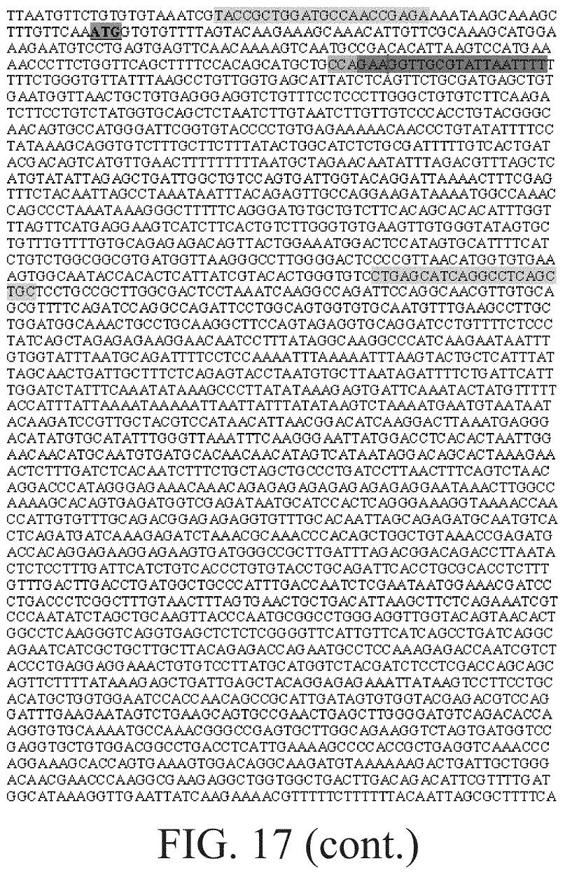

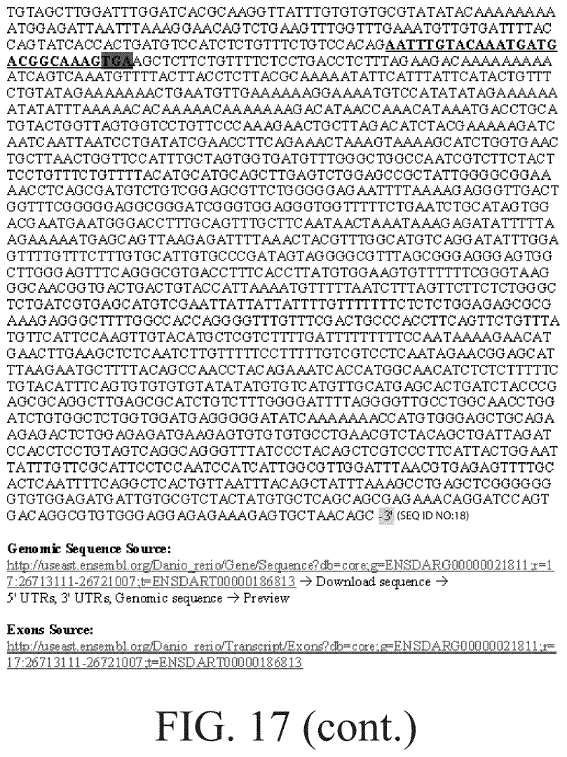

### Example 8: Calculating the Size of the calm1a Target DNA and Cut Target DNA Products (FIG. 17)

Directions: The number of nucleotides can be found by selecting the nucleotides (letters), clicking Review and Word Count, and locating Characters (no spaces).

1. Calculate the size of the Target DNA by selecting the nucleotides in and between the primers.

Size of Target DNA=______bp (Fill in the blank)

2. Calculate the size of the Cut Target DNA product 1 by selecting the first nucleotide of the Forward Primer sequence and all the nucleotides to the cutting location.

Size of Cut Target DNA product 1=______bp (Fill in the blank)

3. Calculate the size of the Cut Target DNA product 2 by subtracting the two numbers from #1 and #2.

Size of Cut Target DNA product 2=______bp (Fill in the blank)

4. Find the Start Codon. Is the cutting location at the beginning, middle, or end of the gene?

5. What is the benefit of cutting the gene at that location?

6. Is the cutting location in an intron or exon?

7. If you were to choose a target sequence, would you choose one that was in an intron or exon, and why?

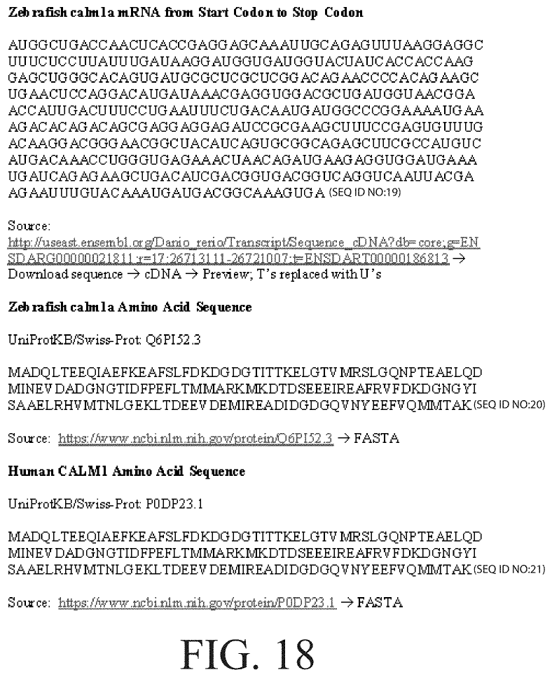

### Example 9: Comparing Zebrafish and Human CALM1 Amino Acid Sequences (FIG. 18)

Directions

1. Go to https://blast.ncbi.nlm.nih.gov/Blast.cgi?PROGRAM=blastp&PAGE_TYPE=BlastSearch&LINK LOC=blasthome.

2. If not already selected, click “Align two or more sequences.” Then, copy and paste the amino acid sequences from FIG. 18.

3. Scroll down, and click on the “BLAST” button.

4. Click on the “Alignments” tab to view the alignment of the sequences.

5. How similar are the calmla (zebrafish) and CALM1 (human) amino acid sequences?

6. Mutations in CALM1 can result in tachycardia (heart beats faster than 100 beats per minute) in humans. What do you predict might happen if you mutated calmla in the zebrafish?

7. Go to the article at https://www.ncbi.nlm.nih.gov/pmc/articles/PMC5886338. Scroll down to Table 1. Name at least one human disease being researched in zebrafish.

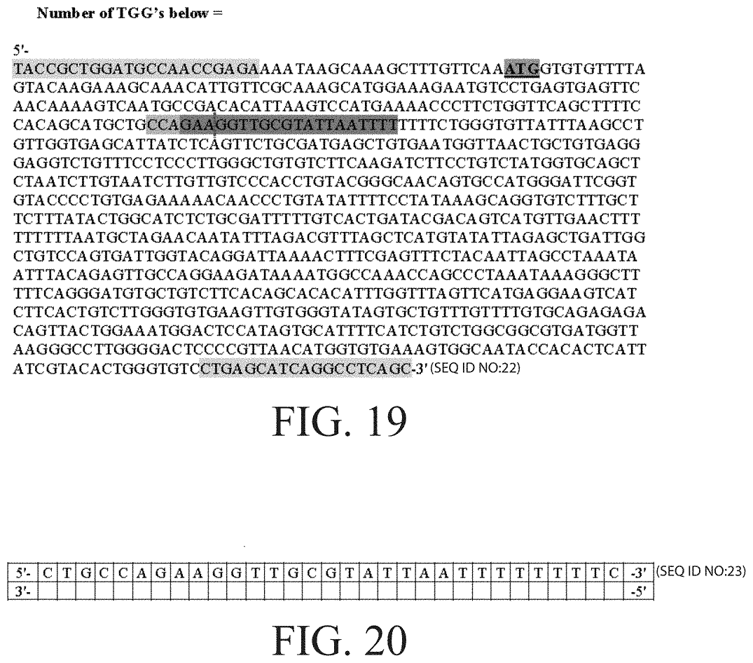

### Example 10: In Vitro Nuclease Assay Activity (FIGS. 19-23)

An in vitro nuclease assay involves a nuclease (Cas9) cutting DNA in a tube. Cas9 is guided by gRNA to a specific location within DNA, where it cuts.

1. Cas9 scans the DNA for the PAM sequence (AGG, CGG, GGG, TGG). Using the Find function, search how many times Cas9 would momentarily stop at TGG when scanning the coding strand below from 5′ to 3′? (See FIG. 19).

2. DNA is double-stranded, but for simplicity, only one strand is usually shown. Part of the fourth row of the coding strand is shown in FIG. 20. Add the noncoding strand via complementary base pairing. (See FIG. 20).

3. In the noncoding strand, search 5′ to 3′ until you find the TGG. Circle that TGG.

4. For Cas9 to cut, the Target Sequence in the DNA needs to come immediately 5′ to the PAM sequence. The bottom strand goes 5′ to 3′ when you read the letters from right to left. Thus, the Target Sequence is just right of the PAM sequence TGG in the FIG. 20 example.

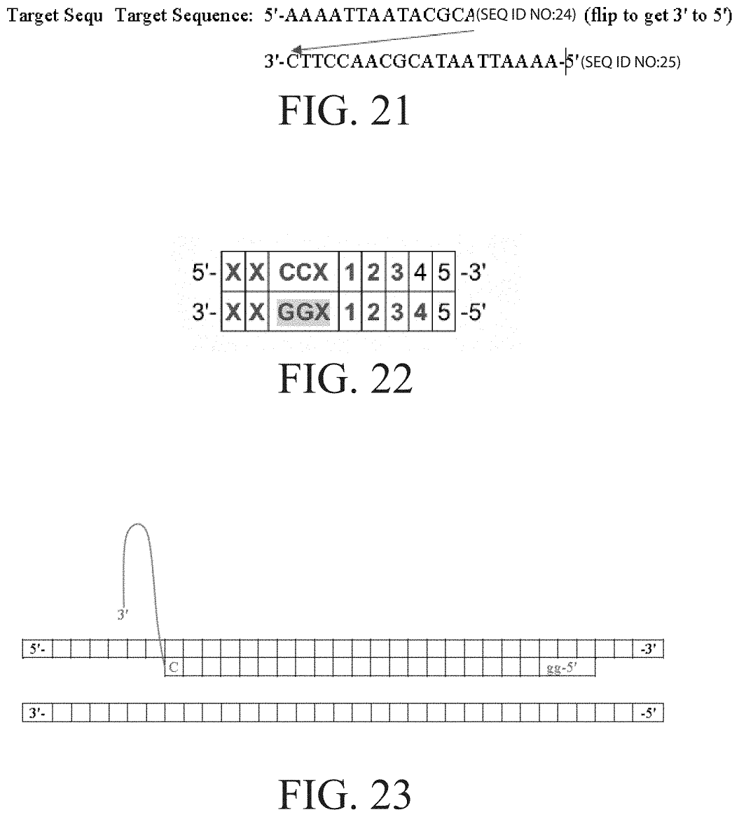

Box the Target Sequence (shown in FIG. 21) in the noncoding strand you created.

5. Researchers have shown that Cas9 cuts between nucleotides 4 and 5 from the PAM site as you go towards the 5′ end of the DNA and between nucleotides 3 and 4 as you go towards the 3′ end on the opposite strand. See the Cas9/gRNA/Target DNA Figure and FIG. 22 for an example. Then, in #2, bold the letters that correspond to the nucleotides that will be part of Cut Target DNA product 1 (the nucleotides to the left of the cutting sites), and change the font to blue.

6. As Cas9 unwinds the two strands of DNA, the Target Sequence within the DNA separates from the sequence complementary to it. That allows the Target Sequence within the gRNA to bind the complementary sequence in the DNA. Cas9 cuts both the complementary sequence and the Target Sequence located in the DNA. Watch the Cas9/gRNA Animation.

Complete the following diagram (see FIG. 23) to include the following:
the two strands of DNA separated; copy and paste the DNA sequences from #2
the gRNA bound to the DNA nucleotides that are complementary to the Target Sequence

(SEQ ID NO: 8)
5′-ggAAAAUUAAUACGCAACCUUC------------------------
----------------------------3′;

Cas9 overlapping the DNA strands and cutting sites and attached to the tail end of the gRNA

### Example 11: Primer-BLAST Activity

Primer-BLAST is a bioinformatics tool used for designing primers, as well as obtaining PCR product size. Once you know how to use such tools, information can be found relatively quickly.

1. Go to Primer-BLAST: https://www.ncbi.nlm.nih.gov/tools/primer-blast.

2. Copy and paste the following information.

Forward Primer Sequence:
(SEQ ID NO: 9)
5′-TACCGCTGGATGCCAACCGAGA-3′
Reverse Primer Sequence:
(SEQ ID NO: 10)
5′-GCAGCTGAGGCCTGATGCTCAG-3′

Organism: Danio rerio (taxid:7955)

Database: Select Genomes . . .

3. Scroll down, and click “Get Primers.”

4. If the results haven't shown within one minute, click “Check”

5. The “Product on target templates” lets you know what gene or part of a gene is being copied during PCR. Click on the link given on the Primer-BLAST site.

In the new tab that opens, search for gene.

Which gene is it?

6. Go back to the Primer-BLAST tab. What is the PCR product size (product length) when using those primers?

7. The annealing temperature (the temperature at which the primers bind the DNA strands) is based on which type of DNA polymerase (the molecular machine that copies DNA) is used. When Taq polymerase is used, the predicted annealing temperature is 5° C. less than the average of the melting temperatures (Tm). Average the melting temperatures of the primers used by adding the numbers and then dividing by 2. Last, subtract 5° C.

predicted annealing temperature=

8. How does the annealing temperature that you used compare to the predicted annealing temperature?

### Example 12: Designing Primers with Benchling

1. Design Primers for the Target DNA (The Target DNA will be a segment of DNA that will include the Target Sequence.)

The strategy is to create an approximately 1,000-bp Target DNA that will be cut to produce two products, such as 300 bp and 700 bp.

a. Click the Target Sequence.

b. Click the Primers icon ( ) “CREATE PRIMERS”, and “Wizard”.

c. For the Region, click “Use Selection”. For the Amplicon, use 700 for the “Min” and 1200 for the “Max”. Then, click “Generate Primers”.

d. Select the first primer pair by checking the box in front of the primers.

e. Click “SEQUENCE MAP” to make sure the Target Sequence isn't within 100 base pairs of either primer. If the primers aren't within 100 base pairs, paste a screenshot below.

f. Click the Forward Primer and drag the right bar to include all of the Reverse Primer.

g. Click “Copy” and the DNA Sequence.

h. Go to Primer-BLAST: https://www.ncbi.nlm.nih.gov/tools/primer-blast.

i. Paste the possible Target DNA sequence into the PCR Template box.

j. In the Primer Parameters section of Primer-BLAST, paste the possible Forward Primer sequence and the possible Reverse Primer sequence in the Primer section. To obtain the Forward and Reverse Sequences in Benchling, click “PRIMER3 RESULTS”, double click on the sequence, right click, and click “Copy”.

k. For Database, select “Genomes for selected organisms . . . ” or “Refseq representative genomes”. For Organism, select the appropriate model organism.

l. Check the box to “Show results in a new window”, and click “Get Primers”.

m. If unintended products (products that aren't part of your gene of interest) are predicted, start the process over with another primer pair. Also, make sure the primers' melting temperatures (Tm) are within 5° C. of each other and the complementarity values are relatively low.

2. Annotate the Target DNA Primers in Benchling

a. Within Benchling in the “PRIMER3 RESULTS”, check the box of the selected primer pair.

b. Click “Save Selected Primers” and “Create”.

c. Close “PRIMER WIZARD” and “PRIMER3 RESULTS”.

d. Click the search icon to search for the Forward Primer sequence.

e. Click the highlighted Reverse Primer sequence.

f. Click “Create”, “Primer”, and “Reverse”. Then, name the primer “Reverse Primer”, and click “Save Primer”.

g. If the Target Sequence is the same color as the primers, click the Target Sequence and then the Annotate icon. Change the color to aqua, and click “Save annotation”. Then, click the Annotate icon again to close the Annotate pop-up window.

A laboratory experience centered around the CRISPR-Cas9 system has the potential to introduce students to a variety of crucial life science topics, including bioinformatics, genetics, and molecular/cellular biology. For example, bioinformatics modules can introduce students to gene annotations, navigating gene browsers, and using sequence analysis tools. Through the in vitro techniques used with this kit, students learn about DNA/RNA synthesis, DNA editing, and analysis by agarose/polyacrylamide gel electrophoresis in a rich application-oriented context. Extensions could include genotyping, Western blotting, pulse field electrophoresis, and immunohistochemistry. Because CRISPR-Cas9 can be applied to any number of organisms (e.g., bacteria, yeast, plant, zebrafish, and mammalian systems), students could explore differences in genome organization, processing of genetic information, and gene conservation. Utilizing CRISPR-Cas9 also allows authentic research opportunities. Students can research genes of unknown function and those that have not yet been fully annotated.

By taking an in vitro approach to CRISPR-Cas9 work, students make and test the tools for genome editing without actually injecting them into organisms or cells. In this manner, they learn the underlying concepts and methodology while circumventing the ethical issues of working with whole animals. In addition, the work provides an opportunity to discuss real and potential ethical, legal, and social impacts (ELSI), and related considerations involving the use of CRISPR-Cas9 technology in crops/animals/humans and the implications on biodiversity and the natural environment.

Certain terminology is used herein for purposes of reference only, and thus is not intended to be limiting. For example, terms such as “upper,” “lower,” “above,” and “below,” refer to directions in the drawings to which reference is made. Terms such as “front,” “back,” “rear,” “bottom,” and “side,” describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first,” “second,” and other such numerical terms referring to structures do not imply a sequence or order unless clearly indicated by the context.

When introducing elements or features of the present disclosure and the exemplary embodiments, the articles “a,” “an,” “the,” and “said,” are intended to mean that there are one or more of such elements or features. The terms “comprising”, “including,” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

The sequence listing txt file in computer readable form, named “3294.006 ST25”, having a size of 21 KB and created on Oct. 27, 2021, is incorporated by reference herein.

What I claim is:

1. A CRISPR-Cas9 kit comprising a sealable package including:
parafilm for mixing the loading dye with DNA thereon;
0.2 milliliter tubes;
1.5 milliliter tubes;
screw-cap tubes;
falcon conical centrifuge tubes; and
reagents selected from a group consisting of: forward primer, reverse primer, gene specific oligo, tail oligo, PCR (master mix), T4 DNA polymerase, dNTPs, NEB buffer 2.1, T7 RNA polymerase, NTPs, buffer, DTT, RiboGuard RNase Inhibitor, DNase I, ammonia acetate, EtOH, RNA storage solution, Cas9, NEBuffer 3.1, proteinase K, agarose, TAE buffer, DNA gel stain, DNA ladder, loading dye, and nuclease-free water.

2. The kit of claim 1, further comprising at least one of:
tube labels;
bag and box labels; and
resealable bags.

3. The kit of claim 2, further comprising at least one of
an insulated container; and
dry ice.

4. A method of performing CRISPR-Cas9 techniques, comprising
providing a kit according to claim 1; and
performing at least one of the following CRISPR-Cas9 techniques:
annealing oligos and making the DNA Template,
gRNA synthesis, DNase I treatment, and RNA precipitation, and
in vitro nuclease assay with CRISPR-Cas9.

5. The method of claim 4, wherein the technique of annealing oligos and making the DNA Template comprises the following steps:
in a PCR tube labeled with group number, “Annealed Oligos”, and date, combining the following:

| | |
|---|---|
| gene-specific oligo (100 μM): | 1.0 μL |
| tail oligo (100 μM): | 1.0 μL |
| nuclease-free water: | 8.0 μL; |

and
placing the PCR tube labeled “Annealed Oligos” in a thermocycler with the following program:
96° C. for 5 mins;
Ramp down 0.1° C. per sec. to 25° C.;
4° C. for infinity.

6. The method of claim 5, wherein the technique of annealing oligos and making a DNA template further comprises the following steps:
transferring 5 μL of the annealed oligos to the PCR tube labeled DNA Template;
transferring 5 μL of the Extension Master Mix (Ext. MM) to the PCR tube labeled DNA Template;
wherein an “Annealed Oligos” tube can be stored at −20° C.;
wherein the Extension Master Mix contains the following:

| | |
|---|---|
| dNTPs (10 mM): | 1.25 μL per reaction |
| NEB Buffer 2.1 (10X): | 1 μL per reaction |
| T4 DNA polymerase: | 1 μL per reaction |
| Nuclease-free water: | 1.75 μL per reaction; |

placing the PCR tube labeled DNA Template in the thermocycler with the following program:
12° C. for 45 mins. (DNA Template is made);
95° C. for 10 sec. (denatures T4 DNA polymerase, but oligos also denature);
Ramp down 0.1° C. per sec. to 25° C. (˜12 mins.; oligos anneal) 4° C. for infinity; and
storing the tube labeled DNA Template tube at −20° C.

7. The method of claim 4, wherein the technique of gRNA synthesis, DNase I treatment, and RNA precipitation comprises the following steps:
transferring 1.5 μL of DNA Template to the “gRNA w/DNA” tube;
transferring 3.5 μL of gRNA MM to the “gRNA w/DNA” tube wherein the gRNA MINI contains the following:

| | |
|---|---|
| T7 RNA Polymerase: | 0.5 μL |
| Transcription Reaction Buffer (10X): | 0.5 μL |
| ATP (100 mM): | 0.5 μL |
| GTP (100 mM): | 0.5 μL |
| CTP (100 mM): | 0.5 μL |
| UTP (100 mM): | 0.5 μL |
| DTT (100 mM; helps inhibit RNases): | 0.5 μL |
| RNase Inhibitor: | 0.12 μL; |

placing the “gRNA w/DNA” tube in a thermocycler with the following program:
38° C. for 30 min.;
to the “gRNA w/DNA” tube, adding 15 μL the DNase I MM (DI MM) for a total reaction volume of 20 μL;
incubating at 37° C. for 30 min.;
transferring 20 μL of gRNA from the “gRNA w/DNA” tube to the gRNA tube;
to the gRNA tube, adding 70 μL of the RNA Prec. MM;
pipetting up and down three times to mix; and
incubating the gRNA tube at −20° C. for 30 min.

8. The method of claim 7, wherein the technique of gRNA synthesis, DNase I treatment, and RNA precipitation further comprises the following steps:
centrifuging the gRNA tube 15 min. at 4° C. at 12,000 rpm;
pouring out supernatant;
transferring 1 mL of RNA Wash (70% EtOH) to the gRNA tube;
centrifuging 5 min at 4° C. at 12,000 rpm;
removing supernatant via pouring and using a P10 pipette with a tip;
drying ˜15 min. at room temperature until pellet is clear;
transferring 30 μL of RNA Storage Solution (1 mM sodium citrate, pH 6.5) to the gRNA tube;
pipetting up and down five times to mix; and
storing at −20° C.

9. The method of claim 4, wherein the technique of in vitro nuclease assay with CRISPR-Cas9 comprises the following steps:
in the Target DNA tube, combining the following:
1 μL of DNA (1,000 ng/μL; from the zebrafish tail fin)
1 μL of Forward Primer (10 micromolar)
1 μL of Reverse Primer (10 micromolar)
22 μL of PCR MM;
after PCR tubes have been prepared, placing the tubes in the designated thermal cycler set with the following program:
Cycle 1: Initial Denaturation: 95° C. for 3 minutes;
Cycle 2: Denaturation: 95° C. for 30 seconds, Annealing: 62° C. for 30 seconds, Extension: 72° C. for 1 minute, and repeating this cycle 35 times;
Cycle 3: Final Extension: 72° C. for 10 minutes;
Cycle 4: Hold at 4° C. for co;
combining 6× Loading Dye+1 μL Target DNA on parafilm and adding to the appropriate lane in a gel;
running the gel for 30 minutes at 120 V; and
viewing the gel in blue or UV light.

10. The method of claim 9, wherein the technique of in vitro nuclease assay with CRISPR-Cas9 further comprises the following steps:
transferring 1 μL of gRNA to the “Cut Target DNA” tube;
transferring 5 μL of the Cas9 MM to the “Cut Target DNA” tube, wherein the Cas9 MM includes the following:

| | |
|---|---|
| Cas9: | 0.6 μL |
| NEBuffer 3.1 (10X): | 1.0 μL |
| Nuclease-free water: | 3.4 μL; |

incubating the “Cut Target DNA” tube at 37° C. for 10 minutes;
to the “Cut Target DNA” tube, adding 4 μL of the Target DNA;
incubating the “Cut Target DNA” tube at 37° C. for 30 minutes;
to the “Cut Target DNA” tube, adding 1 μL of Proteinase K (20 mg/mL); and
incubating at 37° C. for 30 minutes.

11. The method of claim 10, wherein the technique of in vitro nuclease assay with CRISPR-Cas9 further comprises the following steps:
to a 250-mL Erlenmeyer flask adding the following:
50 mL lx TAE buffer;
1 g agarose;
microwaving the agarose for 1.5 minutes in a microwave and removing the flask from the microwave;
adding 5 μL DNA gel stain to the agarose and swirling;
pouring the agarose into the gel-casting tray;
placing the 8-well comb into the gel-casting tray;
once the gel has solidified, removing the comb from the solidified gel;
placing the gel in an electrophoresis chamber with wells facing the negative electrode end;
filling the chamber with 1×TAE to the max fill line;
combining the following:
Lane 1: 2 μL ladder;
On parafilm: 1 μL Loading Dye+2 μL Target DNA;
To the “Cut Target DNA” tube, add 2 μL Loading Dye, pipet up and down 5 times to mix;
running the gel at 120 V for 30 min; and
viewing the gel with blue or UV light.


SEQUENCE LISTING (25 sequences)

SEQ ID NO: 1 - DNA, 22 bp, Artificial Sequence
  calm1a_FP
taccgctggatgccaaccgaga

SEQ ID NO: 2 - DNA, 22 bp, Artificial Sequence
  calm1a_RP
gcagctgaggcctgatgctcag

SEQ ID NO: 3 - DNA, 54 bp, Artificial Sequence
  calm1a_GSO
taatacgactcactataggaaaattaatacgcaaccttcgttttagagctagaa

SEQ ID NO: 4 - DNA, 80 bp, Artificial Sequence
  Tail Oligo
aaaagcaccgactcggtgccactttttcaagttgataacggactagccttattttaacttgctatttctagctctaaaac

SEQ ID NO: 5 - DNA, 54 bp, Artificial Sequence; identical to SEQ ID NO: 3
  Gene-Specific Oligo

SEQ ID NO: 6 - DNA, 80 bp, Artificial Sequence; identical to SEQ ID NO: 4
  Tail Oligo

SEQ ID NO: 7 - DNA, 80 bp, Artificial Sequence
  Reverse Sequence of Tail Oligo
caaaatctcgatctttatcgttcaattttattccgatcaggcaatagttgaactttttcaccgtggctcagccacgaaaa

SEQ ID NO: 8 - RNA, 22 nt, Artificial Sequence
  DNA Sample
ggaaaauuaauacgcaaccuuc

SEQ ID NO: 9 - DNA, 22 bp, Artificial Sequence
  Forward Primer Sequence
taccgctggatgccaaccgaga

SEQ ID NO: 10 - DNA, 22 bp, Artificial Sequence
  Reverse Primer Sequence
gcagctgaggcctgatgctcag

SEQ ID NO: 11 - DNA, 54 bp, Artificial Sequence; identical to SEQ ID NO: 3
  5′-End

SEQ ID NO: 12 - DNA, 80 bp, Artificial Sequence; identical to SEQ ID NO: 7
  Tail Oligo

SEQ ID NO: 13 - DNA, 37 bp, Artificial Sequence
  DNA Coding Strand
ggaaaattaatacgcaaccttcgttttagagctagaa

SEQ ID NO: 14 - DNA, 22 bp, Artificial Sequence
  Forward Primer
taccgctggatgccaaccgaga

SEQ ID NO: 15 - DNA, 22 bp, Artificial Sequence
  Reverse Primer
gcagctgaggcctgatgctcag

SEQ ID NO: 16 - DNA, 22 bp, Artificial Sequence
  Reverse Complement of Reverse Primer
ctgagcatcaggcctcagctgc

SEQ ID NO: 17 - DNA, 20 bp, Artificial Sequence
  Target Sequence
aaaattaatacgcaaccttc

SEQ ID NO: 18 - DNA, 9028 bp, Artificial Sequence
  calm1a
ttctatgtttacttttattgaaatgttgctgatgaataaaaaaaatatttgaaagtttttggttgtgaatatggttataaatgtgccttttgtttgtcgttttcatgtttgaaaagtgtgtatatgttatgatctacttataaaacttattgactgtaagtttattgagaactacaaccgttttcacattatgtatactgaatgttaatatactaaatcgaatgtattagtaacagtttattaagtactccagtgataataacataaaaatacatattgaatacattcatccagctgcttgctatattaatctacttaatatacttttaaatatactgtgcatgttcattgatgaaagtgtgacacatatttcagacctacagaaaatatcatatgtatcatatcatataataaaatgacccaaacagacgtactgtagtccattattaggtttttacataactaaacaacactttcccttcacggcgcttatgataagatgggccggtttacaactgcactatgcggaagtgagtgatgggggccgtacggcggaggaatacatagatgtatatagtaccggatcaccttcaatactctgcgagcgtcagttgcacggtggagctttaacggaggcggagcatcgctgcagtctagagtagattaacaaacccagtccactctccaccctcagacatggtaagtataggcatttatcatacactaatcatttatgaaatcctgtctatcttttatttgtgtacgccttatgaatgttagtatatacatatgatgtctaacgtaaccacctgcctgcctctaacgttactgcgctcgtgcaacattcatcagacgctgtaatgtatttcaatgcactttgcaagcgatgctgcgtccgttattattttcaacgtgttgcggtgttattgatgtcatagtgcggcaggaaatatgacatatgcgttttattatggtgtgatgagctggacagcagcagacagatgatctctgctcactacgttactattttaaaaaatcccattattatttccttcgtgatttacgtgtttgaaaccacgtttgttttgcattagcgaatgatttttgattgagacattattgacagcgtctgtgcaaaatcaggcagtctgaataatgactatagggatatcttaaagaaactgtttattaaataaataataccaatctgttattattcatggaccatcttgttattccagaccttttctttttaactggaacactaataagctgttaggctgaatgacatcctgagtcccccattcgctttcattgtatggaagaagatgctatggaagtgaatggtgattggcgctgtcaggttttgtgttgttccacagaagaatgccaaaaagatgataagttgaatatttgggagatatttcttaaaactgtaatcaatgaggtccatttaagcagctcttatgtgggctgttttggcagtagagcgtcgtttcatttatgaagacaacacaaggcattttacatgtgtgtattaccatgggatttatctgcgatgtacttctactagtttgcacaactgtcggatctagtaaaataagctagattaacaacatacactgacttcattcattggtttatgttggcgaaacatttttgattcattgtcttggaaatgtaacaggtttgtttgttatctggactttaatgttctgtgtgtaaatcgtaccgctggatgccaaccgagaaaataagcaaagctttgttcaaatggtgtgttttagtacaagaaagcaaacattgttcgcaaagcatggaaagaatgtcctgatgagttcaacaaaagtcaatgccgacacattaagtccatgaaaacccttctggttcagcttttccacagcatgctgccagaaggttgcgtattaattttttttctgggtgttatttaagcctgttgggagcattatctcagttctgcgatgagctgtgaatggttaactgctgtgagggaggtctgtttcctcccttgggctgtgtcttcaagatcttcctgtctatggtgcagctctaatcttgtaatcttgtgtcccacctgtacgggcaacagtgccatgggattcggtgtacccctgtgagaaaaacaaccctgtatattttcctataaagcaggtgtctttgcttctttatactggcatctctgcgatttttgtcatgatacgacagtcatgttgaactttttttttaatgctagaacaatatttagacgtttagctcatgtatattagagctgattggctgtccagtgattggtacaggattaaaactttcgagtttctacattagcctaaataatttacagagttgccaggaagataaaatggccaaaccagccctaaataaagggctttttcagggatgtgctgtcttcacagcacacatttggtttagttcatgaggaagtcatctcactgtcttgggtgtgaagttgtgggtatagtgctgtttgttttgtgcagagagacagttactggaaatggactccatagtgcattttcatctgtctggcggcgtgatggttaagggccttggggacccccgttaacatggtgtgaaagtggcaataccacactcattatcgtacactgggtgtcctgagcatcaggcctcagctgctcctgccgcttggcgactcctaaatcaaggccagattccaggcaacgtgtgcagcgttttcagatccaggccagattcctggcagtggtgtgcaatgtttgaagccttgctggatggcaaactgcctgcaaggcttccagtagaggtgcaggatcctgttttctccctatcagcagagagaaggaacaatcctttataggcaaggcccatcaagaataatttgtggtatttaatgcagattttcctccaaaatttaaaaatttaagtactgctcatttattagcaactgattgctttctcaagtacctaatgtgcttaatagattttctgattcatttggatctatttcaaatataaagcccttatataaagagtgattcaaatactatgtttttaccatttattaaaataaaaattaattatttataaagtctaaaatgaatgtaataatacaagatccgttgctacgtccataacattaacggacatcaaggacttaaatgagggacatatgtgcatatttgggttaaatttcaagggaattatggacctcacctaattggaacaacatgcaatgtgatgcacaacaacatagtcataataggacagcactaaagaaactctttgatctcacaatctttctgctagctgccctgatccttaactttcagtctaacaggaccatagggagaaacaaacagagagagagagagagaggaataaacttggccaaaagcacagtgagatggtcgagataatgcatccactcagggaaaggtaaaaccaaccattgtgtttgcagacggagaaggtgtttgcacaattagcagagatgcaatgtcactcagatgatcaaagagatctaaacgcaaacccacagctggctgtaaaccgagatgaccacaggagaaggagaagtgatgggccgcttgatttgacggacagaccttaatactctcctttgattcatctgtcaccctgtgtacctgcagattcacctgcgcacctctttgtttgacttgacctgatggctgcccatttgaccaatctcgaataatggaaagatccctgaccctcggctttgtaactttagtgaactgctgacattaagcttctcagaaatcgtcccaatatctagctgcaagttacccaatgcggcctgggaggttggtacagtaacactggcctcagggtcaggtgagctctctcggggttcattgttcatcagcctgatcaggcagaatcatcgctgcttgcttacagagaccagaatgcctccaaagagaccaatcgtctaccctgaggaggaaactgtgtcttatgcatggtctacgatctcctcgaccagcagcagttcttttataaagagctgattgagctacaggagagaaattataagtccttcctgcacatgctggtggaatccaccaacagccgcattgatgtgtggtacgagacgtccaggatttgaagaatagtctgaagcagtgccgaactgagcttggggatgtcagacaccaaggtgtgcaaaatgccaaacgggccgagtgcttggcagaaggtctagtgatgtccgaggtgctgtggacggcctgacctcattgaaaagccccaccgctgaggtcaaacccaggaaagcaccagtgaaagtggacaggcaagatgtaaaaaagactgattgctgggacaacgaacccaggcgaagaggctggtggctgacttgacagacattcgttttgatggcataaaggttgaattatcaagaaaacgtttttcttttttacaattagcgcttttcaacaaccttttaaggagatactatcagtttttaaattctaggtcaaggttgtcttggtttattgagtttcaaagagaaagttttgaatatcactataaagtgacagggcaaatcaaaaacattcatgatctaatgagatgttatgttcgtgttatggttgagtttttcctctgttccttttttacattgcacactctggatatgcatttgttggttccagcttttttgacgcttgtttttcttcatgacgggacattctgccattttaggtttttctcagtttgacgctaatgcgcttggcacaacaaaatgatgacttgtatgtgtttatgacactcatttactcatcacttttcttcttctccatttacaggctgaccaatcaccgaggagcaaattgcaggtaaggcagtctgtttgtggtgtgaacataccctgaattagcaccagtaccagaataatggtcgcaatattagcttcttacactctgacgtttcaaatatttgaggttaaaccctatctttctgtttcctcacagagtttaaggaggctttctccttatttgataaggatggtgatggtactatcaccaccaaggagctgggcacagtgatgcgctcgctcggacagaacccccagaagctgaactccaggacatgataaacgaggtggacgctgatggtgagttgagttcatgacaatcagcgatgctgtctgtaagaatgtgctatattaaggttggggttaagtgttttttaaagcgattgtaggcagtcagcacacaaacaatagaaacgctttcctggcacatctgtacacacaacgctggtttgcaaggctgatttttggcctatcctgctctggtatggttctactcgaaagcttcctctccctattgagaacgggtgtttaaacgttgattaaaccgttatcttgttttgtaattagcttgtgtgcaagcaagcaagctttcaaatagcaccaaatgtaatacgcctgggatgcctctcttgttacgagctgatgtaattagtctttcagtatgctgtggtgtctttagagaatgtcctttaacacttgtattgttagatcatgcagagtttctgagacatactgatcattttcagatgggattgtgcgctatgagtcgtttttgtaggtcagacagcagtaaagcccgttgaataacagcaggtaataaagacctcatggaatcctttacataatccactttggcagtggtggcagtggaatggagagtgtttgtgaaggcttcagtgagaagaacatgcagtgtgaagaaaaacacacacacacacacacacacatacataagcacgtgtctacacccactttgtcagattacacgcgcagtgtgagccctatgggaatggctgtgtgatcattttcacacacgcatgcacacaggaggatgtcatatttgtcccgtgtaatggaaatatcccacctcagcttggttaaacctcaagggaagcaaaacacgggcatgtgtgcgcacacacaccacacacttcagcaggactgtctcatctttcctgtattgtttagatttcaattagctactattttgtttttggaatataattatacacacaaatggcttcaatttaaattctgcaataagtttaactagtaggtttaacataactttaaaacctacaaccttttttaaatgctataagggaatttggacatcaccacatattattgtctagtatgaaaagagatttaagtgtttttaaacacactttaaaggaggtacgatgagagcataggtactcaaagtaaaaatggaggatatcaagcatgctgttcatgcctgtgacttcgcctttgtagatttgtatgcatcagtgagactgtcaactagggccgatttagttcactagatgatacccaaacagacttaatacatgctattagctctcattacgcctcattatactctgtttactaggctgctcttctccttcactcttggatactccaccaggttttcccaaacacaatagctacatcagataaatgtctgttgttttcaggtaacggaaccattgactttcctgaatttctgacaatgatggcccggaaaatgaaagacacagacagcgaggaggagatccgcgaagctttccgagttttgacaaggtcagtggtgaaaagaggggcattgtgggaagtatttctgctgccatggaaagagtgaggaggcaggaaaaaacaaaccaacgctcggaaaactaaataacaagcccttgttccatataagctgtcattggtttcaagcagatggcccagtcagagatttttccatgtttacagagacatttgattgtcaatggcctctcttgcttccttttgtaaaatgttaccagtgccttctctttttaatcccattatgccttcgcaacacatcattttttttgcaacctgctttggatttagtgcttttaatatttgaacagcctgacttattgatcctgtctgtctctgtcaggacgggaacggctacatcagtgcgcagagcttcgccatgtcatgacaaacctgggtgagaaactaacagatgaagaggtggatgaaatgatcagagaagctgacatcgacggtgacggtcaggtcaattacgaaggtacgattcagctgttgctgagattacctggaaagagtttttttttttttttttttaaatgcagattaaacattacttttaggaattgccacttccaattataataatgtagcttggatttggatcacgcaaggttatttgtgtgtgcgtatatacaaaaaaaaatggagattaatttaaaggaacagtctgaagtttggtttgaaatgttgtgattttaccagtatcaccactatgtccatctctgtttctgtccacagaatttgtacaaatgatgacggcaaagtgaagctcttctgttttctcctgacctctttagaagacaaaaaaaaaatcagtcaaatgttttacttacctcttagcaaaaatattcatttattcatactgtttctgtatagaaaaaaactgaatgttgaaaaaaggaaaatgtccatatatagaaaaaaatatatttaaaaacacaaaaacaaaaaaagacataaccaaactaaatgacctgcatgtactggttagtggtcctgttcccaaagaactgcttagacatctacgaaaaagatcaatcaattaatcctgatatcgaaccttcagaaactaaagtaaaagcatctggtgaacgcttaactggttccatttgctagtggtgatgtttgggctggccaatcgtcttctacttcctgtttctgttttacatgcatgcagcttgagtctggagccgctattggggcggaaaacctcagcgatgctgtcggagcgttctgggggagaattttaaaagagggttgactggtttcgggggaggcgggatcgggtggagggtggtttttctgaatctgcatagtggacgaatgaatgggacctttgcagtttgctcaataactaaataaagagatatttttaagaaaaatgagcagttaagagattttaaactacgtttggcatgtcaggatatttggagttttgtttctttgtgcattgtgcccgatagtaggggcgtttgcgggagggagtggcttgggagtttcagggcgtgacctttcaccttatgtggaagtgttttttcgggtaagggcaacggtgactgactgtaccattaaaatgtttttaatctttagttcttctctggctctgatcgtgagcatgtcgaattattattattttgtttttttctctctggagagcgcgaaagagggcttttggccaccaggggtttgtttcgactgcccaccttcagttctgtttatgttcattccagttgtacatgctcgtcttttgatttttttttccaataaaagaacatgaacttgaagctctcaatcttgtttttcctttttgtcgtcctcaatagaacggagcatttaagaatgcttttacagccaactacagaaatcaccatggcaacatctctctttttctgtacatttcagtgtgtgtgtatatatgtgtcatgttgcatgagcactgatctacccgagcgcaggcttgagcgcatctgtctttggggatttaggggttgcctggcaacctggatctgtggctctggtggatgagggggatatcaaaaaaaccatgtgggagctgcagaagagactctggagagatgaagagtgtgtgtgcctgaacgtctacagctgttagatccacctcctgtagtcaggcagggtttatccctacagctcgtcccttcattactggaattatttgttcgcattcctccaatccatcattggcgttggatttaacgtgagagttttgcactcattttcaggctcactgttaatttacagctatttaaagcctgagctcgggggggtgtggagatgattgtgcgtctactatgtgctcagcagcgagaaacaggatccagtgacaggcgtgtgggaggagaaaagagtgctaacagc

SEQ ID NO: 19 - RNA, 447 nt, Artificial Sequence
  calm1a
auggcugaccaacucaccgaggagcaaauugcagaguuuaaggaggcuuucuccuuauuugauaaggauggugaugguacuaucaccaccaaggagcugggcacagugaugcgcucgcucggacagaccccacagaagcugaacuccaggacaugauaaacgagguggacgcugaugguaacggaaccauugacuuuccugaauuucugacaaugauggcccggaaaaugaaagacacagacagcgaggaggaguccgcgaagcuuuccgaguguuugacaaggacgggaacggcuacaucagugcggcagagcuucgccaugucaugacaaaccugggugagaaacuaacagaugaagagguggaugaaaugaucagagagcugacaucgacggugacggucaggucaauuacgaagaauuuguacaaaugaugacggcaaaguga

SEQ ID NO: 20 - protein, 148 aa, Artificial Sequence
  UniProtKB/Swiss-Prot Q6PI52.3
MADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIRADIDGDGQVNYEEFVQMMTAK

SEQ ID NO: 21 - protein, 148 aa, Artificial Sequence; identical to SEQ ID NO: 20
  UniProtKB/Swiss-Prot Q6PI52.3

SEQ ID NO: 22 - DNA, 944 bp, Artificial Sequence
  DNA sample
taccgctggatgccaaccgagaaaataagcaaagctttgttcaaatggtgtgttttagtacaagaaagcaaacattgttcgcaaagcatggaaagaatgtcctgagtgagttcaacaaaagtcaatgcgacacattaagtccatgaaaacccttctggttcagcttttccacagcatgctgccagaaggttgcgtattaattttttttctgggtgttatttaagcctgttggtgagcattatctcagttctgcgtgagctgtgaatggttaactgctgtgagggaggtctgtttcctcccttgggctgtgtcttcaagatcttcctgtctatggtgcagctctaatcttgtaatcttgttgtcccacctgtacgggcaacatgccatgggattcggtgtacccctgtgagaaaaacaaccctgtatattttcctataaagcaggtgtctttgcttctttatactggcatctctgcgatttttgtcactgatacgacagtcatgttgaatttttttttaatgctagaacaatatttagacgtttagctcatgtatattagagctgattggctgtccagtgattggtacaggattaaaactttcgagtttctacaattagcctaaataatttacagattgccaggaagataaaatggccaaaccagccctaaataaagggctttttcagggatgtgctgtcttcacagcacacatttggtttagttcatgaggaagtcatcttcactgtcttgggtgtgaagtttgggtatagtgctgtttgttttgtgcagagagacagttactggaaatggactccatagtgcattttcatctgtctggcggcgtgatggttaagggccttggggactccccgttaacatggtgtgaaatggcaataccacactcattatcgtacactgggtgtcctgagcatcaggcctcagc

SEQ ID NO: 23 - DNA, 31 bp, Artificial Sequence
  DNA Sample
ctgccagaaggttgcgtattaattttttttc

SEQ ID NO: 24 - DNA, 20 bp, Artificial Sequence
  Noncoding Strand
aaaattaatacgcaaccttc

SEQ ID NO: 25 - DNA, 20 bp, Artificial Sequence
  Noncoding Strand
cttccaacgcataattaaaa